(19) United States
(12) Patent Application Publication
(10) Pub. No.: US 2019/0259499 A1
(43) Pub. Date: Aug. 22, 2019

(54) SYSTEM AND METHOD FOR PREDICTING SEQUENTIAL ORGAN FAILURE ASSESSMENT (SOFA) SCORES USING ARTIFICIAL INTELLIGENCE AND MACHINE LEARNING

(71) Applicant: Peach IntelliHealth Pte Ltd., Trivex (SG)

(72) Inventors: L.S. Klaudyne Hong, New York, NY (US); Gerald Wogan, Belmont, MA (US); Luigi Vacca, Weston, MA (US); Bruce Tidor, Lexington, MA (US)

(21) Appl. No.: 16/342,127

(22) PCT Filed: Oct. 18, 2017
(86) PCT No.: PCT/IB2017/001433

§ 371 Date: Apr. 15, 2019

(51) Int. Cl. G16H 50/20; G16H 10/60; G16H 50/30; G06N 20/00; A61B 5/00

(52) U.S. Cl. CPC G16H 50/20; G16H 10/60; A61B 5/72; G06N 20/00; G16H 50/30

(57) ABSTRACT

Various aspects of the subject technology related to systems and methods for predicting sequential organ failure assessment (SOFA) scores using machine learning. A system may be configured to receive patient data including one or more features associated with one or more patients. The system may process the features using one or more SOFA score prediction models derived from at least one machine learning process to output respective predicted SOFA scores. One of the prediction models has been trained to output a first SOFA component score for a first amount of time into the future and a second prediction model has been trained to output a second SOFA component score for the first amount of time into the future. The system may output on a graphical user interface, a total SOFA score, the first SOFA component score, and the second SOFA components score predicted for the respective patient.

### RELATED APPLICATIONS

The present application claims priority to the U.S. Provisional Application No. 62/410,249 filed on Oct. 19, 2016 and titled “USE OF CLINICAL PARAMETERS FOR THE PREDICTION OF PATIENT MORTALITY AND ORGAN FAILURE,” which is herein incorporated by reference in its entirety.

### BACKGROUND

Sequential organ failure assessment (SOFA) is a clinical evaluation and scoring method used for determining the state of a patient's organ function or rate of organ failure during hospitalization, for example while a patient is being treated in an intensive care unit (ICU) of a hospital. SOFA scores are strongly correlated with patient mortality which enables healthcare practitioners to employ SOFA scoring as a good predictor of patient death. SOFA scoring can be performed in an iterative manner throughout a patient's hospital stay to follow the course of organ dysfunction as a patient's health deteriorates. SOFA scores also show a strong correlation in determining the prognosis and likelihood of patient mortality due to a variety of other conditions including sepsis, influenza, tuberculosis, liver disease, respiratory and cardiovascular diseases. SOFA scoring has also been useful in demonstrating the effects of various therapeutic interventions the patient may have received.

The total SOFA score is based on six individual SOFA component scores. The SOFA scoring method includes one SOFA component scores for the respiratory, cardiovascular, hepatic, coagulation, renal and neurological organ systems. Each SOFA component score is determined based on point-giving conditions that are associated with specific measured values of one or more clinical parameters or medications related to the particular SOFA components score organ system being evaluated. The total SOFA score is a sum of the six SOFA component scores.

To compute the hepatic SOFA component score, the patient's bilirubin levels are measured and a SOFA component score is assigned as follows: for a bilirubin level between 20 and 32 μmol/L, the SOFA component score is 1; for a level between 33 and 101, the SOFA component score is 2; for a level between 102 and 204 the SOFA component score is 3; and for a level >204, SOFA component score is 4.

To compute the neurological SOFA component score, the Glasgow coma scale (GCS) is used as follows: for a GCS value between 13 and 14 inclusive, the SOFA component score is 1; for a GCS value between 10 and 12 inclusive, SOFA component score is 2; for a GCS value between 6 and 9, the SOFA component score is 3; and for any GCS value less than 6, the SOFA component score is 4.

To compute the respiratory SOFA component score, the measured ratio between PAO2 (arterial partial pressure) and FIO2 (fraction of inspired oxygen) is used as follows: for a ratio between 300 and 400, the SOFA component score is 1; for a ratio between 200 and 300, the SOFA component score is 2; for a ratio between 100 and 200, the SOFA component score is 3; and for a ratio between 0 and 100, the SOFA component score is 4.

To compute the coagulation SOFA component score, the number of platelets divided by 10³/μl is used as follows: for a platelet value between 100 and 150, the SOFA component score is 1; for a platelet value between 50 and 100, the SOFA component score is 2; for a platelet value between 20 and 50, the SOFA component score is 3; and for a platelet value less than 20, the SOFA component score is 4.

To compute the renal SOFA component score, the patient's creatinine levels are measured and a SOFA component score is assigned as follows: for a creatinine level between 110 and 170, the SOFA component score is 1; for a creatinine level between 171 and 299, the SOFA component score is 2; for a creatinine level between 300 and 440, the SOFA component score is 3; and for a creatinine level greater than 440, the SOFA component score is 4.

The cardiovascular SOFA component score uses the MAP (mean arterial pressure) and/or the administered vasopressor dosage information (e.g., dopamine, dobutamine, epinephrine, and norepinephrine) as follows: if MAP was less than 70, the cardiovascular SOFA component score is 1; for administration of a dopamine dosage less than 5μg/kg/min or administration of any dobutamine dose, the cardiovascular SOFA component score is 2; for administration of a dopamine dose greater than 5 μg/kg/min or administration of an epinephrine dose less than or equal to 0.1 μg/kg/min or administration of a norepinephrine dose less than or equal to 0.1 μg/kg/min, the cardiovascular SOFA component score is 3; and for administration of a dopamine dosage greater than 15 μg/kg/min or administration of an epinephrine dose greater than 0.1 μg/kg/min or administration of a norepinephrine dose greater than 0.1 μg/kg/min, the cardiovascular SOFA component score is 4.

Machine learning is an application of artificial intelligence that automates the development of an analytical model by using algorithms that iteratively learn patterns from data without explicit indication of the data patterns. Machine learning is commonly used in pattern recognition, computer vision, email filtering and optical character recognition and enables the construction of algorithms that can accurately learn from data to predict model target outputs thereby making data-driven predictions or decisions.

The description provided in the background section should not be assumed to be prior art merely because it is mentioned in or associated with the background section. The background section may include information that describes one or more aspects of the subject technology.

### SUMMARY

According to one aspect, the disclosure relates to a computer-implemented method for predicting sequential organ failure assessment (SOFA) scores using machine learning. The method includes receiving patient data including a plurality of features associated with one or more patients. The method also includes processing the plurality of features for each patient using a plurality of SOFA score prediction models derived from at least one machine learning process to output a plurality of respective predicted SOFA scores. A first of the prediction models has been trained to output a first SOFA component score for a first amount of time into the future and a second of the prediction models has been trained to output a second SOFA component score for the first amount of time into the future. The method further includes outputting on a graphical user interface, for each of the patients, a total SOFA score and at least one of the first SOFA component score and the second SOFA component score predicted for the respective patient.

In some implementations, the method includes determining the total SOFA score for each patient via a third prediction model trained to output total SOFA scores for the first amount of time into the future. In some implementations, the method includes calculating the total SOFA score for each patient by summing the values of six SOFA component scores for a given patient for first amount of time into the future, wherein each of the SOFA component scores is associated with a different organ system. In some implementations, the method includes determining a second total SOFA score for each patient by via a fourth prediction model trained to output total SOFA scores for a second amount of time into the future. In some implementations, the method includes processing the patient data for each patient using a SIRS score prediction model derived from a machine learning process to output a predicted SIRS score, where the SIRS score prediction model has been trained to output a value indicating the likelihood of a patient having at least two SIRS symptoms the first amount of time into the future and outputting on a graphical user interface, for each of the patients, the SIRS score along with the total SOFA score and at least one of the first SOFA component score and the second SOFA component score predicted for the respective patient. In some implementations, each of the first and second SOFA component scores correspond to a different one of a respiratory organ system, a cardiovascular organ system, a hepatic organ system, a coagulation organ system, a renal organ system, and a neurological organ system. In some implementations, the method includes processing a subset of the plurality of features to estimate a current value of a physiological parameter for a patient. The physiological parameter is a physiological parameter used in calculating a current SOFA component score. In some implementations, the method includes processing a subset of the plurality of features to predict a future value of a physiological parameter for a patient score for the first amount of time into the future. The physiological parameter is a physiological parameter traditionally used in calculating a SOFA component score. In some implementations, the method includes outputting on the graphical user interface an indication for at least one patient of any SOFA component scores predicted to exceed a threshold value, an identification of the organ system associated with the SOFA component score exceeding the threshold value, and the amount of time in the future at which the SOFA component score is predicted to exceed the threshold. In some implementations, the method includes outputting on the graphical user interface a list of patients for whom any SOFA component score is predicted to exceed a threshold value the first amount of time in the future.

According to certain aspects of the present disclosure, a system for predicting sequential organ failure assessment (SOFA) scores using machine learning is provided. The system includes a memory storing computer-readable instructions and a plurality of SOFA score prediction models. The system also includes a processor configured to execute the computer-readable instructions. The instructions, when executed cause the processor to receive patient data including a plurality features associated with one or more patients. The processors are further configured to process the plurality of features for each patient using a plurality of SOFA score prediction models derived from at least one machine learning process to output a plurality of respective predicted SOFA scores. A first of the prediction models has been trained to output a first SOFA component score for a first amount of time into the future and a second of the prediction models has been trained to output a second SOFA component score for the first amount of time into the future. The processors are configured to output on a graphical user interface, for each of the patients, a total SOFA score and at least the first SOFA component score and the second SOFA component score predicted for the respective patient.

In some implementations, the memory is further configured to store computer-readable instructions, which when executed cause the processor to determine the total SOFA score for each patient via a third prediction model trained to output total SOFA scores for the first amount of time into the future. In some implementations, the memory is further configured to store computer-readable instructions, which when executed cause the processor to calculate a total SOFA score for each patient by summing the values of six SOFA component scores for the patient for first amount of time into the future, wherein each of the SOFA component scores is associated with a different organ system. In some implementations, the memory is further configured to store computer-readable instructions, which when executed cause the processor to determine a second total SOFA score for each patient via a fourth prediction model trained to output total SOFA scores for a second amount of time into the future. In some implementations, the memory is further configured to store computer-readable instructions, which when executed cause the processor to carry out the method further including processing the patient data for each patient using a plurality of SIRS score prediction models derived from at least one machine learning process to output a predicted SIRS score, where the plurality of SIRS score prediction models have been trained to output a SIRS score for one or more amounts of time into the future and outputting on a graphical user interface, for each of the patients, the SIRS score in addition to the total SOFA score, the first SOFA component score and/or the second SOFA component score predicted for the respective patient. In some implementations, each of the first and second SOFA component scores correspond to a different one of a respiratory organ system, a cardiovascular organ system, a hepatic organ system, a coagulation organ system, a renal organ system, and a neurological organ system. In some implementations, the memory is further configured to store computer-readable instructions, which when executed cause the processor to carry out the method further including processing a subset of the plurality of features to estimate a current value of a physiological parameter for a patient. The physiological parameter is a physiological parameter used in calculating a current SOFA component score. In some implementations, the memory is further configured to store computer-readable instructions, which when executed cause the processor to carry out the method further including processing a subset of the plurality of features to predict a future value of a physiological parameter for a patient score for the first amount of time into the future. The physiological parameter is a physiological parameter traditionally used in calculating a SOFA component score. In some implementations, memory is further configured to store computer-readable instructions, which when executed cause the processor to output on the graphical user interface the total SOFA score and the first and second SOFA component scores and displaying an indication of the first and second SOFA component scores exceeding a threshold value, wherein the graphical output identifies the organ system associated with the SOFA component score exceeding the threshold value. In some implementations, the memory is further configured to store computer-readable instructions, which when executed cause the processor to output on the graphical user interface the total SOFA score and the first and second SOFA component scores and displaying an indication identifying a list of patients whose total SOFA score and first or second SOFA component scores exceeds a threshold value.

According to certain aspects of the present disclosure, a system for predicting a total sequential organ failure assessment (SOFA) score is provided. The system includes a memory storing computer-readable instructions and a total SOFA score prediction model. The system also includes a processor configured to execute computer-readable instructions. The instructions, when executed, cause the processor to receive patient data including a plurality of features associated with one or more patients. The processors are further configured to process the plurality of features for each patient using a total SOFA score prediction model derived from at least one machine learning process to output a predicted total SOFA score for the patient for a first amount of time into the future. The total SOFA score prediction model takes as input the patient's current values of at least three physiological parameters, including a Braden Score and at least two of Glasgow Coma Scale, platelet level, and creatinine level. The processors are further configured to output on a graphical user interface, for each of the patients, the total SOFA scores predicted for the respective patients for the first amount of time into the future.

In some implementations, the system includes a total SOFA score prediction model which takes as input the patient's current values of a Braden Score, platelet level, creatinine level, and the Glasgow Coma Scale. In some implementations, the system includes a total SOFA score prediction model further takes as input the patient's current values of at least two of albumin level, heart rate, and age. In some implementations, the system includes a total SOFA score prediction model including a support vector regression model. In some implementations, the system includes a total SOFA score prediction model including a radial basis function support vector regression model. In some implementations, the memory is further configured to store computer-readable instructions, which when executed cause the processor to carry out the method further including determining the total SOFA score for each patient via a second prediction model trained to output a total SOFA score for a second amount of time into the future, different than the first amount of time into the future. In some implementations, the memory is further configured to store computer-readable instructions, which when executed cause the processor to carry out the method further including determining a future value of one or more SOFA component scores for each patient predicted for the first amount of time into the future. In some implementations, the memory is further configured to store computer-readable instructions, which when executed cause the processor to carry out the method further including, for each patient, displaying a SOFA component score predicted for the first amount of time into the future. In some implementations, the memory is further configured to store computer-readable instructions, which when executed cause the processor to carry out the method further including outputting on the graphical user interface an indication of at least one predicted physiological parameter value associated with the predicted SOFA component score.

According to certain aspects of the present disclosure, a method for predicting a total sequential organ failure assessment (SOFA) score is provided. The method includes receiving patient data including a plurality of features associated with one or more patients. The method also includes processing the plurality of features for each patient using a total SOFA score prediction model derived from at least one machine learning process to output a predicted total SOFA score for the patient for a first amount of time into the future. The total SOFA score prediction model takes as input the patient's current values of at least three physiological parameters, including a Braden Score and at least two of Glasgow Coma Scale, platelet level, and creatinine level. The method further includes outputting on a graphical user interface, for each of the patients, the total SOFA scores predicted for the respective patients for the first amount of time into the future.

In some implementations, the method includes processing the plurality of features for each patient using a total SOFA score prediction model which takes as input the patient's current values of a Braden Score, platelet level, creatinine level, and the Glasgow Coma Scale. In some implementations, the method includes processing the plurality of features for each patient using a total SOFA score prediction model further takes as input the patient's current values of at least two of albumin level, heart rate, and age. In some implementations, the method includes processing the plurality of features for each patient using a total SOFA score prediction model including a support vector regression model. In some implementations, the method includes processing the plurality of features for each patient using a total SOFA score prediction model including a radial basis function support vector regression model. In some implementations, the method further includes determining the total SOFA score for each patient via a second prediction model trained to output a total SOFA score for a second amount of time into the future, different than the first amount of time into the future. In some implementations, the method further includes determining a future value of one or more SOFA component scores for each patient predicted for the first amount of time into the future. In some implementations, the method further includes, for each patient, displaying a SOFA component score predicted for the first amount of time into the future. In some implementations, the method further includes outputting on the graphical user interface an indication of at least one predicted physiological parameter value associated with the predicted SOFA component score.

According to certain aspects of the present disclosure, a computer readable storage medium containing program instructions for causing a computer to predict sequential organ failure assessment (SOFA) scores using machine learning is provided. The program instructions contained on the computer readable storage medium perform the method including receiving patient data including a plurality features associated with one or more patients. The program instructions further perform the method including processing the plurality of features for each patient using a plurality of SOFA score prediction models derived from at least one machine learning process to output a plurality of respective predicted SOFA scores. A first of the prediction models has been trained to output a first SOFA component score for a first amount of time into the future and a second of the prediction models has been trained to output a second SOFA component score for the first amount of time into the future. The program instructions further perform the method including outputting on a graphical user interface, for each of the patients, a total SOFA score and at least the first SOFA component score and the second SOFA component score predicted for the respective patient.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates an example architecture for predicting SOFA scores using machine learning.

FIGS. 2A-2C illustrate example block diagrams of systems for predicting SOFA scores using machine learning according to some implementations.

FIG. 3 is a flowchart showing a method for predicting SOFA scores using a SOFA score prediction model derived from a machine learning process.

FIGS. 4A-4D illustrate example user interfaces for displaying and interacting with patients predicted SOFA scores on small-format computing devices according to some implementations.

FIGS. 5A-5D illustrate example user interfaces for displaying and interacting with patients predicted SOFA scores on large-format computing devices according to some implementations.

FIG. 6 is a block diagram of an example computing system.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The disclosed system and method provides for predicting SOFA scores for a patient at a specified amount of time into the future using a prediction model trained in a machine learning process. The system and method enable the use of combinations of seemingly unrelated clinical patient data, physiological features, and therapeutic procedures to accurately predict a total SOFA score and SOFA component scores for a patient at specified amounts of time into the future. Traditionally SOFA scores, and specifically SOFA component scores, have been determined based on measuring one or more specific clinical data parameters for a patient and assigning points based on the value of the specific measured clinical parameter. The SOFA component scores are determined for a single instance of time based on the current or most recently measured values of the specific clinical parameters that are used to determine the SOFA component score. By summing the six SOFA components scores, a total SOFA score may be generated for the patient. Similarly, the total SOFA score represents the assessment of the health and condition of a patient's organ systems in the immediate or present time. Because the total SOFA score is strongly correlated to patient morbidity and mortality, healthcare practitioners are able to assess a patient's likelihood of death or further morbidity as determined at the single point of present time in which the SOFA score was determined. While useful as an instantaneous diagnostic assessment tool of a patient's conditions, the ability to predict SOFA scores for a patient at specified amounts of time into the future would assist healthcare practitioners in taking necessary actions to treat and prevent predicted organ system failures which could lead to patient mortality. Additionally, predicting and monitoring SOFA scores for patients at specified amounts of time into the future may enable healthcare practitioners to identify patients who may or may not tolerate certain treatments or interventions, and subsequent drug titration and/or preventative methods applied in regard to the treatment or interventions. For example, monitoring the predicted SOFA scores for a cancer patient may identify the need to decrease the administration of a specific chemotherapeutic to the patient, or to initiate the administration of an additional approved drug(s) in order to moderate future symptoms of the patient as a participant in a clinical trial.

As the complexity and costs associated with modern healthcare systems rise, patients and healthcare practitioners seek new diagnostic or preventative treatment techniques to better predict future events or adverse patient conditions that may, if untreated, lead to patient death or worsening patient morbidity. Healthcare practitioners routinely collect a wide variety of clinical data measurements to characterize one or more aspects of a patient's condition. The collected clinical data measurements provide insight into the immediate condition of the patient as indicated by the collected measurement. While useful in providing insight into the present condition of the patient, these measurements are not always helpful in providing healthcare practitioners with insight about the future condition of a patient's morbidity or risk of mortality. The problem for healthcare practitioners (and patients) is how to accurately determine a patient's likelihood of increased morbidity, disease complications or even risk of death in future amounts of time based on a wide variety of clinical data measurements that are available for measurement in the present amount of time. This problem includes related challenges for healthcare practitioners such as how best to allocate medical personnel, treatment options, and/or specific diagnostic devices or procedures in order to adequately treat or even prevent potentially worsening health conditions that a patient may experience at future amounts of time.

A solution to this problem is proposed employing a machine learning process to train predictive models that are capable of predicting patient SOFA scores at specified amounts of time into the future by using a wide variety of clinical data measurements for the patient, which are measured in the present, as inputs to a trained prediction model. The benefit of this solution provides healthcare practitioners with greater insight about future patient health conditions such that healthcare practitioners can be better prepared to treat patients appropriately based on the anticipated, predicted changes in a patient's morbidity or likelihood of mortality.

A machine learning process can be utilized to generate a trained prediction model capable of determining SOFA scores at future specified amounts of time based on a variety of received patient data. To generate the trained prediction model, a machine learning process iteratively inputs selected subsets of patient data features as training data to a machine learning algorithm. The selected subsets of patient data features may include a wide variety of patient data so that the machine learning algorithm is trained to predict SOFA scores not solely based on the patient data that is routinely used to determine a patient's SOFA scores. For example, the selected subsets of patient data features on which the machine learning algorithm is trained may include nitric oxide, testosterone, and/or magnesium levels. These subsets of patient data features are not traditionally used to determine SOFA scores, but in the machine learning process these features may be used to train the machine learning algorithm to compute a patient's SOFA scores at future amounts of time. As the machine learning algorithm iteratively processes various subsets of patient data features in the training data input, the control parameters of the machine learning algorithm are adjusted to optimize the predictive performance of the algorithm for generating SOFA scores based on the selected subset of patient data features used as training input. After adjusting the control parameters of the machine learning algorithm and completing the learning for a range of subsets of patient data features, the resulting machine learning algorithm can be output as a new trained prediction model. Additional details of the machine learning process used herein to generate one or more predictive models capable of predicting SOFA scores at specified amounts of time into the future can be found below in the “Machine Learning Process Description” section, below.

Once a new prediction model has been trained in the machine learning process, the new prediction model can be deployed, for example stored in memory or configured on the processor of a computing device in a hospital setting, and used to predict a patient's SOFA scores at specified amounts of time into the future. The deployed prediction model may receive a variety of patient data as inputs and determine a total SOFA score and/or one or more SOFA component scores based on the input patient data. The determined SOFA scores can be output in a variety of scenarios or configurations including outputting the SOFA scores to memory, outputting the SOFA scores to a computing device for display on a user interface, or outputting the SOFA scores to a database, such as a patient records database.

The output SOFA scores identifying a patient's predicted SOFA scores at various specified amounts of time into the future may be used by healthcare practitioners in the planning and administration of preventative treatment options to reduce the likelihood of increased risk of patient morbidity or mortality based on the predicted changes in a patient's conditions determined to occur at future time points as indicated by the output SOFA scores. Systems and methods for performing a machine learning process to train a machine learning algorithm and generate a trained prediction model capable of generating a patient's predicted SOFA scores at specified amounts of time into the future will now be described further.

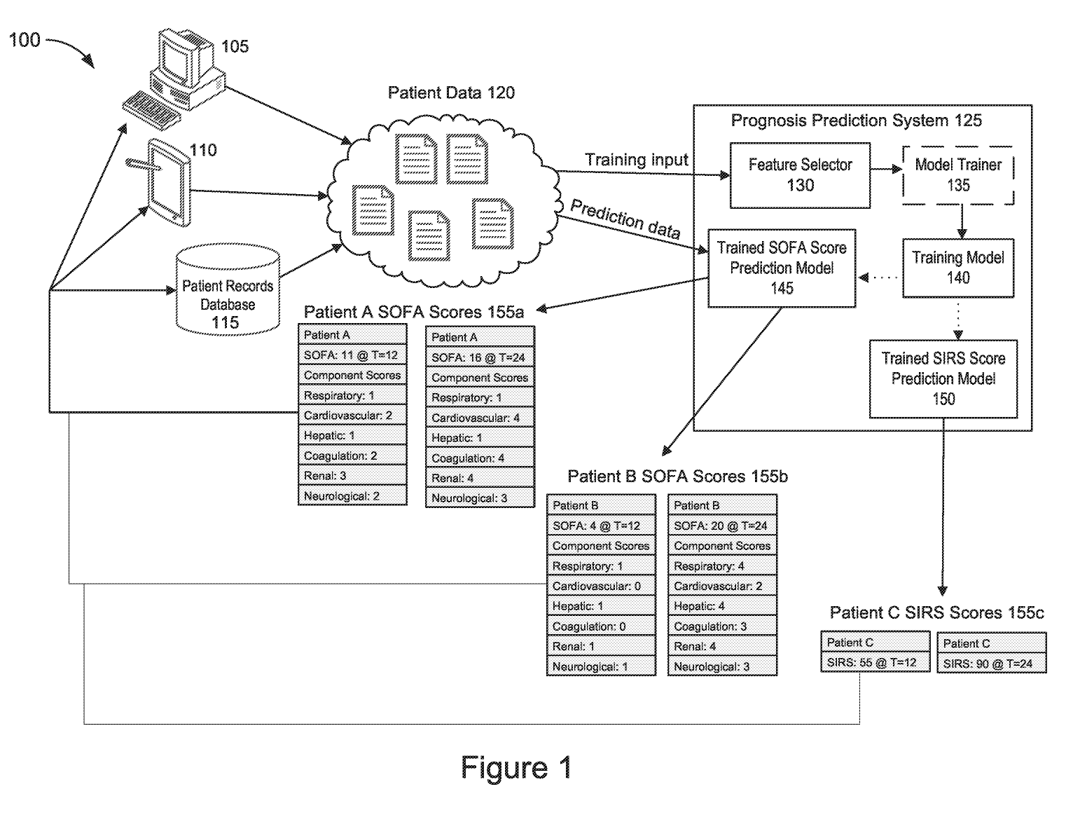

FIG. 1 illustrates an example architecture 100 for predicting SOFA scores using machine learning. The architecture 100 includes a large-format computing device 105 and a small-format computing device 110. The architecture 100 also includes a patient records database 115 and patient data 120. The architecture 100 includes a prognosis prediction system 125, which includes a feature selector 130, a model trainer 135, a training model 140, a trained SOFA score prediction model 145 and a trained SIRS score prediction model 150. The architecture also includes patient scores 155, such as Patient A's SOFA scores 155a, Patient B's SOFA scores 155b, as well as patient C's SIRS scores 155c.

As shown in FIG. 1, a large-format computing device 105 or any other fully functional computing device, such as a desktop computers or laptop computers, may transmit patient data 120 to machine learning trainer 125. Additionally, or alternatively, other computing devices, such as a small-format computing device 110 may also transmit patient data 120 to machine learning trainer 125. Small-format computing device 110 may include a tablet, smartphone, personal digital assistant (PDA), or any other computing device that may have more limited functionality compared to large-format computing devices 105. Patient data may be stored in a database, for example in a patient records database 115 to be transmitted to machine learning trainer 125. Large-format computing device 105 and small-format computing device 110 may include memory storing data and applications related to determining patient SOFA scores. In some implementations, the large-format computing device 105 and the small-format computing device 110 may receive patient data input by healthcare practitioners, other computing devices, or directly from patient monitoring equipment and may transmit the patient data to a prognosis prediction system 125.

As shown in FIG. 1, patient data 120 is transmitted to a prognosis prediction system 125. In some implementations, the patient data 120 includes training input that is transmitted to a prognosis prediction system 125 for use in a machine learning process. The training input is used to train a machine learning algorithm in a machine learning process in order to generate a training model capable of predicting SOFA scores based on a wide variety of received patient data. In other implementations, the patient data 120 includes prediction data that is transmitted to a prognosis prediction system 125 as inputs to the generated model that was trained in the machine learning process using the training input. The patient data 120 may include data that can be utilized in the instant determination of patient's SOFA scores as well as in the prediction of SOFA scores at various amounts of time into the future. For example, the patient data may include a number of standard clinical parameters or measurements, also known as features, which are commonly collected and available in healthcare settings. The features may include common patient measurements, vital signs or observations, laboratory test results, medications taken and the dosage of those medications, as well as any materials, solids, fluids entering and leaving the patient by specified routes. Patient data 120 may be processed in a machine learning process to derive a prediction model that has been trained to output predicted SOFA scores, for a given patient, at a specified amount of time into the future.

As further shown in FIG. 1, architecture 100 includes a prognosis prediction system 125 to receive patient data 120 and output patient SOFA scores, e.g., Patient A's SOFA scores 155a and Patient B's SOFA scores 155b. In broad overview, the prognosis prediction system 125 functions in the training aspect of a machine learning process to receive patient data as training input and generate a training model for use in SOFA score prediction. The prognosis prediction system 125 includes a feature selector 130 which is used in the training aspect of the machine learning process to select subsets of features in the patient data. The prognosis prediction system 125 also includes a model trainer 135 which uses a selected machine learning algorithm to process the selected subsets of features as inputs and generate a new training model 140 which may be subsequently used outside of the machine learning process to predict future SOFA score values based on the received prediction data. Additional details of the machine learning process used herein to generate one or more predictive models capable of predicting SOFA scores at specified amounts of time into the future can be found below in the “Machine Learning Process Description” section, below.

As shown in FIG. 1, the prognosis prediction system 125 includes a feature selector 130. During the training aspect of the machine learning process, the feature selector 130 receives patient data and selects subsets of features in the patient data which are used as training input to train the selected machine learning algorithm. For each selected subset of features in the training input, the selected machine learning algorithm can be trained to predict future SOFA scores associated with the subset of features for which the selected machine learning algorithm was trained. The trained machine learning algorithm can then be output as a new trained model (e.g., training model 140) which may then be subsequently applied to an individual patient's data (e.g., prediction input) to determine SOFA scores predicted at specified amounts of time into the future.

The prognosis prediction system 125 includes a model trainer 135 (as shown in dashed lines). In some implementations, the model trainer 135 may be included in the prognosis prediction system 125. In other implementations, the model trainer 135 may be located remotely from the prognosis prediction system 125. During the training aspect of the machine learning process, the model trainer 135 receives the training input including the selected subsets of features from the feature selector 130 and iteratively applies the subsets of features to the previously selected machine learning algorithm to assess the performance of the algorithm. As the machine learning algorithm processes the training input, the model trainer 135 learns patterns in the training input that map the machine learning algorithm variables to the target output data (e.g., the predicted SOFA scores) and generates a training model that captures these relationships. For example, as shown in FIG. 1, the model trainer 135 outputs the training model 140. As further shown in FIG. 1 using dotted arrow lines, the training model 140 that is output can be a trained SOFA score prediction model 145. In some implementations, a machine learning SIRS trainer (not shown) may output a training model 140 that can be a trained SIRS score prediction model, such as the trained SIRS score prediction model 150.

As further shown in FIG. 1, the prognosis prediction system 125 includes a trained SOFA score prediction model 145. The trained SOFA score prediction model 145 is a model that has been generated as a result of the model training performed during the training aspect of the machine learning process. Once trained, the trained SOFA score prediction model 145 may operate outside of a machine learning process to receive patient data 120 as prediction data and generate patient scores 155 for a given patient. For example, the trained SOFA score prediction model 145 outputs Patient A's SOFA scores 155a. Patient A's SOFA scores 155a include data identifying the predicted SOFA scores at two amounts of time into the future (e.g., T=12, which identifies the SOFA scores predicted 12 hours into the future and T=24, which identifies the SOFA scores predicted 24 hours into the future). The outputted SOFA scores include the total SOFA score predicted at each specified amount of time into the future. For example, Patient A is predicted to have a total SOFA score of 11 at 12 hours into the future. As further shown in Patient A's SOFA scores 155a, Patient A's total SOFA score will rise to 16 at 24 hours into the future. Similarly, the outputted SOFA scores include SOFA component scores. Each SOFA component score is respectively associated with one of the 6 organ systems that are evaluated in the total SOFA score. The SOFA component scores are predicted for each specified amount of time into the future. Patient B's SOFA scores 155b similarly identify the total SOFA score and SOFA component scores as shown for Patient A, but are predicted based on the received patient data 120 for Patient B.

As shown in FIG. 1, the prognosis prediction system 125 includes a trained SIRS score prediction model 150. In some implementations, the prognosis prediction system 125 may be a SIRS prognosis prediction system (not shown). The SIRS prognosis prediction system may similarly function in the training aspect of a machine learning process to receive training input and generate a training model 140 such as a SIRS score prediction model, for example the trained SIRS score prediction model 150. Once trained, the trained SIRS score prediction model 150 may operate outside of a machine learning process to receive patient data 120 as prediction data and generate SIRS scores for a given patient, for example Patient C's SIRS Scores 155c. A SIRS score is the likelihood (or in some cases the probability), expressed in a scale of 0-100, that a patient will experience at least two SIRS symptoms at a specified amount of time into the future. Similar to the outputted SOFA scores for Patient A and Patient B, Patient C's SIRS score 155c include the SIRS score predicted at specified amounts of time into the future (e.g., T=12, which identifies the SIRS score predicted 12 hours into the future and T=24, which identifies the SIRS score predicted 24 hours into the future).

As further shown in FIG. 1, Patient A's SOFA scores 155a, Patient B's SOFA scores 155b, and the SIRS scores 155c are transmitted to the large-format computing device 105, the small-format computing device 110 and/or the patient records database 115. The received patient scores 155 may be output to a graphical user interface on the large-format computing device 105 and/or the small-format computing device 110. The output patient scores 155 may be utilized by healthcare providers to determine patient treatment actions based on the SOFA and SIRS scores predicted for a specified amount of time into the future. In some implementations, the SOFA and SIRS scores predicted for a specified amount of time into the future are stored in the patient records database 115.

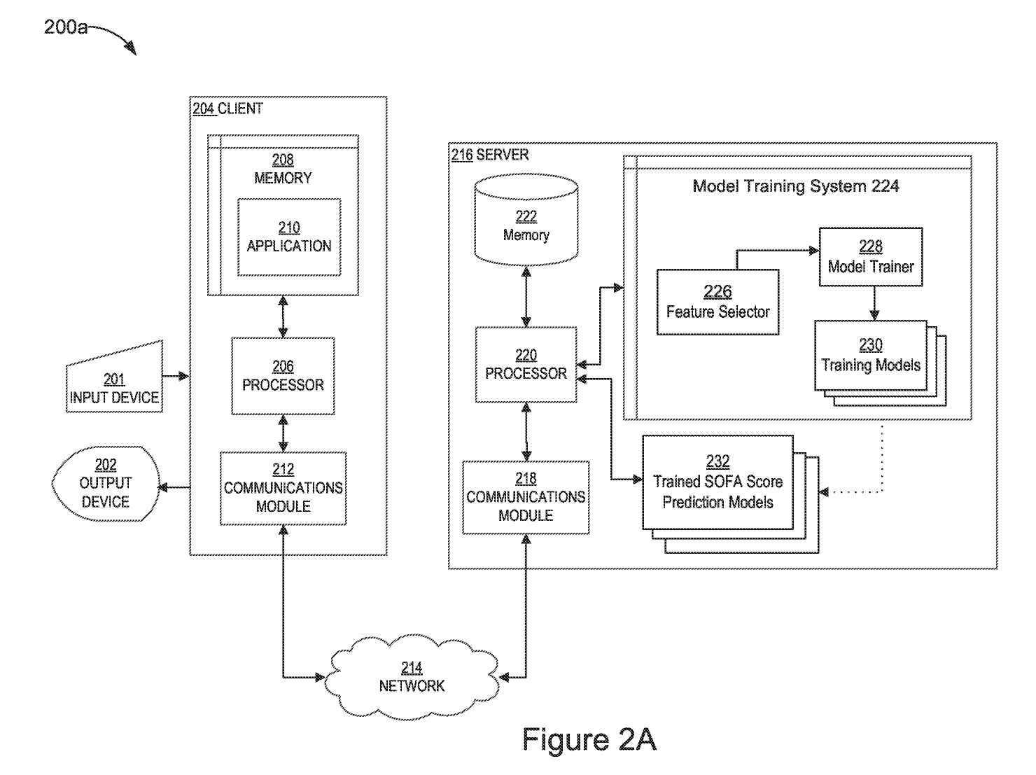

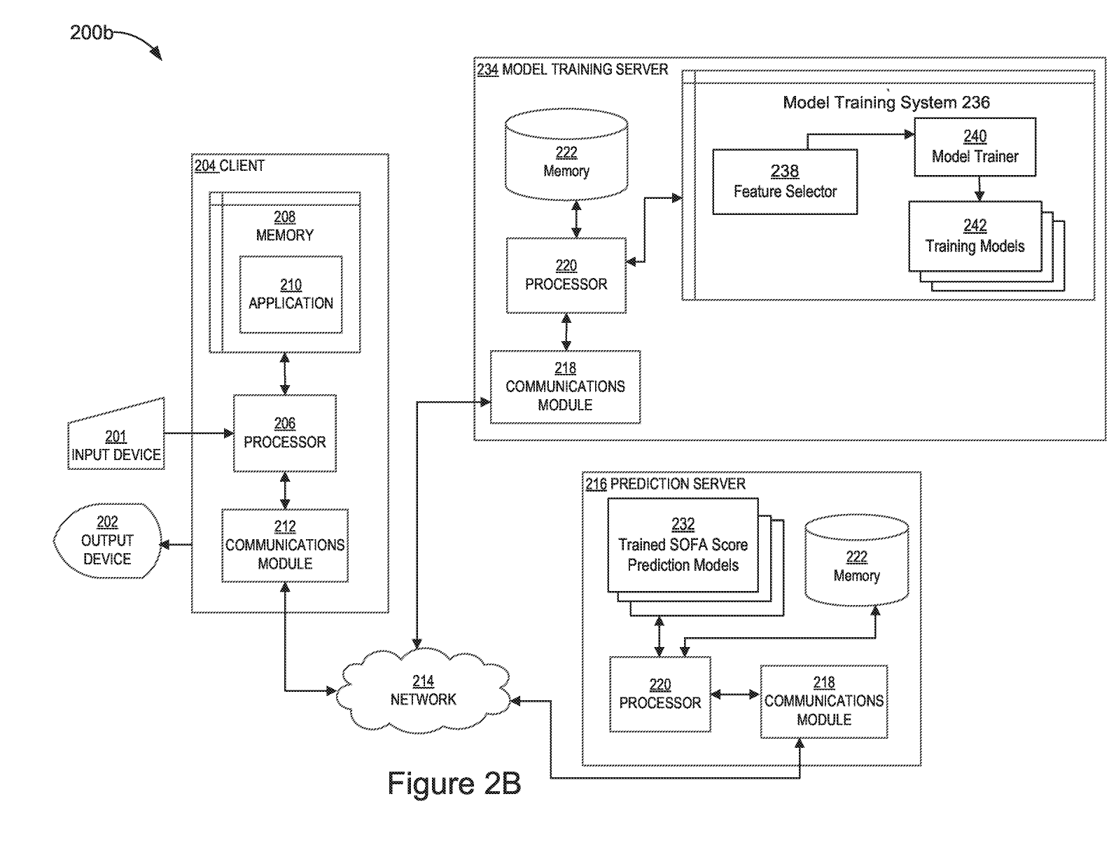

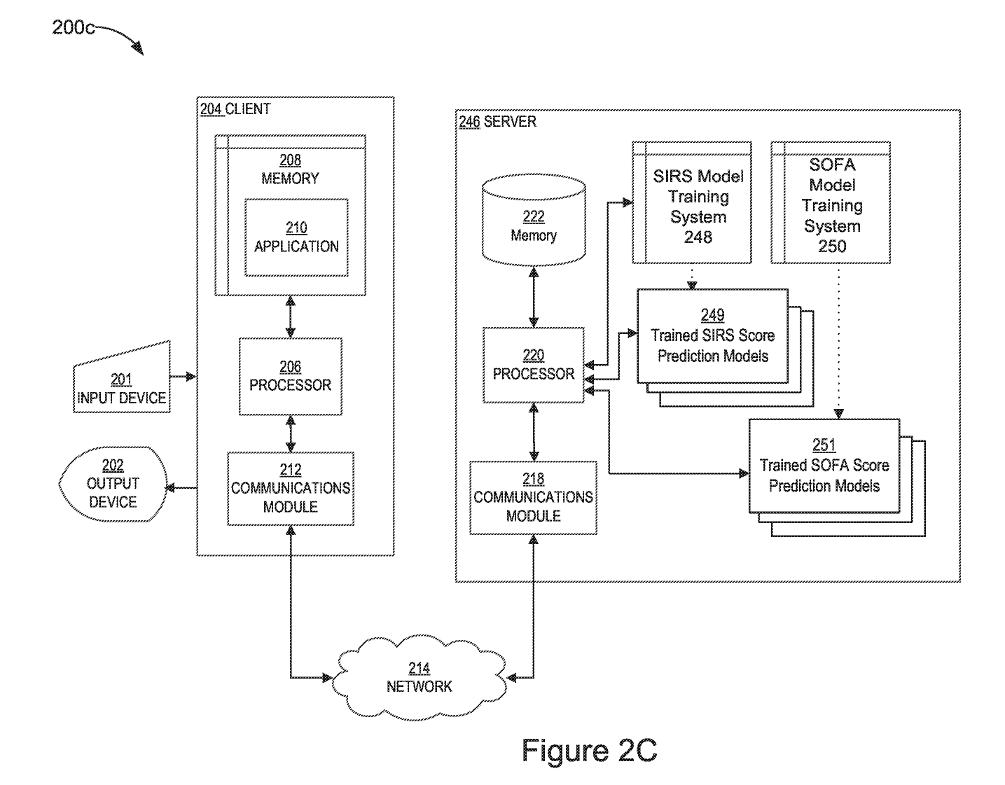

FIG. 2A is an example block diagram of a system 200a for predicting SOFA scores using machine learning according to some implementations. System 200a includes an input device 201 and an output device 202 coupled to a client 204. The client 204 includes a processor 206 and a memory 208 storing an application 210. The client 204 also includes a communications module 212 connected to network 214. System 200a also includes a server 216 which further includes a communications module 218, a processor 220 and a memory 222. The server 216 also includes a model training system 224. The model training system 224 includes a feature selector 226, a model trainer 228 and one or more training models 230. The model training system 224 includes similar components and performs similar operations as the prognosis prediction system 125 shown in FIG. 1, except where indicated otherwise in the foregoing description. The server 216 also includes one or more trained SOFA score prediction models 232, which as described in relation to FIG. 1 are shown in dotted lines to indicate that the training models 230, that were output during the training performed in the machine learning process, can be one or more trained SOFA score prediction models, such as the one or more trained SOFA score prediction models 232.

As shown in FIG. 2A, the system 200a includes an input device 201. The input device 201 receives user input and provides the user input to client 204. The input device 201 may include a keyboard, mouse, microphone, stylus, and/or any other device or mechanism used to input user data or commands to an application on a client, such as client 204. In some implementations, the input device 201 may include haptic, tactile or voice recognition interfaces to receive the user input, such as on a small-format device.

The system 200a also includes a client 204. The client 204 communicates via the network 214 with the server 216. The client 204 receives input from the input device 201. The client 204 can be, for example, a large-format computing device, such as large-format computing device 105 as shown in FIG. 1; a small-format computing device (e.g., a smartphone or tablet), such as small-format computing device 110 also shown in FIG. 1; a medical data device (e.g., a small or large-format device used in a healthcare setting to collect, manage or generate patient diagnostic data or patient record data), or any other similar device having appropriate processor, memory, and communications capabilities. The client 204 may be configured to receive, transmit, and store data associated with predicting SOFA and/or SIRS scores for a patient at various amounts of time into the future.

As further shown in FIG. 2A, the client 204 includes a processor 206 and a memory 208. The processor 206 operates to execute computer-readable instructions and/or data stored in memory 208 and transmit the computer-readable instructions and/or data via the communications module 212. The memory 208 may store computer-readable instructions and/or data associated with predicting a patient's SOFA scores for a specified amount of time into the future. For example, the memory 208 may include a database of patient data, such as patient records database 115 as shown in FIG. 1. The memory 208 includes an application 210. The application 210 may be, for example, an application to receive user input or patient data for use in determining predicted SOFA scores for a given patient at a specified amount of time into the future. In some implementations, the application 210 may receive user input or patient data for use in determining a predicted SIRS score for a given patient at a specified amount of time into the future. The application 210 may include textual and graphical user interfaces to receive patient data as input and display output including predicted SOFA scores for a given patient at one or more amounts of time into the future. The application 210 may include a number of configurable settings associated with triggering alerts or user notifications when a particular patient's SOFA scores or SIRS score exceeds a threshold value. In some implementations, the application 210 may output an indication, in a graphical user interface, identifying a particular organ system associated with a SOFA component score predicted to exceed a threshold value at a specified amount of time into the future. Additionally, or alternatively, the application 210 may output an indication, in a graphical user interface, identifying the amount of time in the future at which a SOFA component score for a given patient is expected to exceed the threshold value. In some implementations, the application 210 may output a list of patients for whom any SOFA component score is predicted to exceed a threshold value for a specified amount of time into the future.

As shown in FIG. 2A, the client 204 includes a communications module 212. The communications module 212 transmits the computer-readable instructions and/or patient data stored on or received by the client 204 via network 214. The network 214 connects the client 204 to the server 216. The network 214 can include, for example, any one or more of a personal area network (PAN), a local area network (LAN), a campus area network (CAN), a metropolitan area network (MAN), a wide area network (WAN), a broadband network (BBN), the Internet, and the like. Further, the network 214 can include, but is not limited to, any one or more of the following network topologies, including a bus network, a star network, a ring network, a mesh network, a star-bus network, tree or hierarchical network, and the like.

As further shown in FIG. 2A, the server 216 operates to receive, store and process the computer-readable instructions and/or patient data generated and received by client 204. In some implementations, the server 216 may receive patient data directly from one or more patient monitoring devices. The server 216 can be any device having an appropriate processor, memory, and communications capability for hosting a machine learning process. In certain aspects, one or more of the servers 216 can be located on-premises with client 204, or the server 216 may be located remotely from client 204, for example in a cloud computing facility or remote data center. The server 216 includes a communications module 218 to receive the computer-readable instructions and/or patient data transmitted via network 214. The server 216 also includes one or more processors 220 configured to execute instructions that when executed cause the processors to determine predicted SOFA scores for a given patient at a specified amount of time into the future. The server 216 also includes a memory 222 configured to store the computer-readable instructions and/or patient data associated with predicting SOFA scores for a given patient at a specified amount of time into the future. For example, memory 222 may store one or more training models, such as the trained SOFA score prediction models 232 generated during the training of a machine learning process which have been trained to output predicted SOFA scores for a patient at various amounts of time into the future. In some implementations, memory 222 may store one or more training models, such as SIRS score prediction models that were similarly generated during a machine learning process and were trained to output predicted SIRS scores for a patient at various amounts of time into the future. In some implementations, the memory 222 may store one or more machine learning algorithms that will be used to generate one or more training models. In some implementations, the memory 222 may store patient data that is received from client 204 and is used as a training dataset in the machine learning process in order to train a SOFA score prediction model and/or a SIRS score prediction model. In some implementations, the memory 222 may store one or more trained prediction models that are used to predict a total SOFA score and one or more training models that are used to predict SOFA component scores.

As shown in FIG. 2A, the server 216 includes a model training system 224. The model training system 224 functions in a machine learning process to receive patient data as training input and processes the patient data to train one or more training models. The model training system 224 includes a feature selector 226, a model trainer 228, and one or more training models 230. In some implementations, the training models 230 that are generated and output as a result of the machine learning process are configured on server 216 as standalone components on server 216. For example, the trained SOFA score prediction models 232 are configured on server 216 to process patient data and output a patient's predicted SOFA scores for specified amounts of time into the future. In some implementations, the trained SOFA score prediction models 232 are stored in memory 222 on server 216.

The model training system 224 is configured to implement a machine learning process which will receive patient data as training input and generate a training model that can be subsequently used to predict SOFA scores at specified amounts of time into the future. The components of the machine learning process operate to receive patient data as training input, select unique subsets of features within the patient data, use a machine learning algorithm to train a model based on the subset of features in the training input and generate a training model that may be output and used for future predictions based on a variety of received patient data. Additional details of the machine learning process used herein to generate one or more predictive models capable of predicting SOFA scores at specified amounts of time into the future can be found below in the “Machine Learning Process Description” section, below.

As shown in FIG. 2A, the model training system 224 includes a feature selector 226. The feature selector 226 operates in the machine learning process to receive patient data and select a subset of features from the patient data which will be provided as training inputs to a machine learning algorithm. In some implementations, the feature selector 226 may select a subset of features corresponding to a SOFA component score such that the machine learning algorithm will be trained to predict a total SOFA score and/or one or more SOFA component scores based on the selected subset of features. In other implementations, the feature processor 226 may select different subsets of features which do not correspond to patient data commonly used to determine a patient's total SOFA score and/or one or more SOFA component scores. By using a variety of training inputs, the machine learning process will generate a trained model that is able to predict a patient's total SOFA score and/or one or more SOFA component scores from a wide variety of disparate patient data.

During the machine learning process, the feature selector 226 provides the selected subset of features to the model trainer 228 as inputs to a machine learning algorithm to generate one or more training models. A wide variety of machine learning algorithms may selected for use including algorithms such as support vector regression, ordinary least squares regression (OLSR), linear regression, logistic regression, stepwise regression, multivariate adaptive regression splines (MARS), locally estimated scatterplot smoothing (LOESS), ordinal regression, Poisson regression, fast forest quantile regression, Bayesian linear regression, neural network regression, decision forest regression, boosted decision tree regression, artificial neural networks (ANN), Bayesian statistics, case-based reasoning, Gaussian process regression, inductive logic programming, learning automata, learning vector quantization, informal fuzzy networks, conditional random fields, genetic algorithms (GA), Information Theory, support vector machine (SVM), Averaged One-Dependence Estimators (AODE), Group method of data handling (GMDH), instance-based learning, lazy learning, and Maximum Information Spanning Trees (MIST).

The model trainer 228 evaluates the machine learning algorithm's prediction performance based on patterns in the received subset of features processed as training inputs and generates one or more new training models 230. The generated training models, e.g., trained SOFA score prediction models 232, are then capable of receiving patient data outside of the machine learning process in which they were trained and generated to output predicted SOFA scores at a specified amount of time into the future for a given patient.

As further shown in FIG. 2A, the trained SOFA score prediction models 232 that were generated as a result of performing the machine learning process, may receive patient data and process the patient data to output predicted SOFA scores to the processor 220. For example, the trained SOFA score prediction models 232, that were produced in the machine learning process, may be subsequently be included in an artificial intelligence system or application configured to receive patient data as prediction inputs and process the data to output SOFA score predictions for a patient at specified amounts of time into the future. In some implementations, the processor 220 may store the predicted SOFA scores output from the trained SOFA score prediction model 232 in memory 222. In some implementations, the memory 222 may store instructions to adjust or transform the received patient data based on the parameter input requirements of trained SOFA score prediction model. For example, if the trained SOFA score prediction model 232 requires that the parameter inputs for calculating the SOFA component score for the coagulation organ system be computed using the number of platelets per 10³/μl (microliters) and the received coagulation patient data identifies the number of platelets per 10³/cc (cubic centimeters), the feature selector 226 would convert the received coagulation patient data to the format required for use in trained SOFA score prediction model to more accurately predict the coagulation SOFA component score. In other implementations, the outputted SOFA scores may be forwarded to communications module 218 for transmission to the client 204 via network 214. Once received by the client 204, the outputted SOFA scores may be transmitted to output device 202, such as a monitor, printer, portable hard drive or other storage device. In some implementations, the output device 202 may include specialized clinical diagnostic or laboratory equipment that is configured to interface with client 204 and may display the predicted SOFA scores in conjunction with the diagnostic or laboratory data for which the specialized clinical diagnostic or laboratory equipment is normally configured to output.

FIG. 2B illustrates an example block diagram of a system 200b using a machine learning process configured on a model training server 234. The individual components and functionality of each component shown and described in relation to model training server 234 in FIG. 2B are identical to the components and respective functionality shown and described in relation to server 216 of FIG. 2A with the exception that the model training server 234 shown in FIG. 2B does not include one or more trained SOFA score prediction models 232 as shown in FIG. 2A.

Instead, as shown in FIG. 2B, the system 200b includes a model training server 234 that is separate from a prediction server 216. The prediction server 216 includes components and functionality similar to the server 216 shown in FIG. 2A with the exception that the prediction server 216 shown in FIG. 2B does not include a model training system, such as the model training system 224 shown in FIG. 2A. The prediction server 216 shown in FIG. 2B includes one or more trained SOFA score prediction models 232.

The trained SOFA score prediction models 232 configured on the prediction server 216 are models that were generated from a machine learning process, such as training models 242 and have been trained in the machine learning process to output predicted SOFA scores for a patient at one or more specified amounts of time into the future. For example, upon receiving patient data from a client, for example client 204, the trained SOFA score prediction models 232 may be employed to generate one or more SOFA scores for a patient at respective amounts of time into the future based on the received patient data. In some implementations, each of the trained SOFA score prediction models 232 may generate a SOFA component score or a total SOFA score for a specific amount of time into the future. In some implementations, each of the trained SOFA score prediction models 232 may generate a SOFA component score or a total SOFA score for a shorter amount of time into the future or a longer amount of time into the future. For example, a first trained SOFA score prediction model 232 may generate SOFA component scores which are predicted 6, 12, or 24 hours in the future, while a second trained SOFA score prediction model 232 may generate a total SOFA score which is predicted 36, 48, or 72 hours into the future.

As shown in FIG. 2B, system 200b also includes a model training server 234. The model training server 234 includes a model training system 236 which implements a machine learning process and includes a feature selector 238, a model trainer 240, and one or more training models 242. In some implementations, the training server 234 may be located in the same location as prediction server 216. In other implementations, the model training server 234 may be located in a remote location, for example in a second data center that is separately located from the data center or hospital premises where the prediction server 216 is located. In some implementations, the model training system 236, configured on the model training server 234, may be utilized to evaluate different machine learning algorithms and generate one or more alternate training models 242. For example, based on using different subsets of features in the received patient data as the training inputs to a different machine learning algorithm and process, the model training system 236 may train and output a different training model 242 than the trained SOFA score prediction models 232 configured on prediction server 216 which may have been trained using a separate machine learning algorithm and process.

The model training system 236 may also be configured with a machine learning process to train and output one or more training models 242 that are capable of generating a total SOFA score and SOFA component scores which are estimated for the current or present time. In some implementations, the model training system 236 may generate a model, such as trained model 222 which may be capable of estimating a current total SOFA score or a current SOFA component score when one or more of the physiological parameters which are traditionally used to determine a particular SOFA component score and total SOFA score are not available. For example, a patient's renal SOFA component score is traditionally calculated based on the patient's measured creatinine levels. If a healthcare practitioner is unable to ascertain or measure a patient's creatinine levels, a model may be generated to output an estimated current creatinine level for use in determining the patient's current renal SOFA component score based on other available physiological parameters. Such physiological parameters can be determined through a machine learning process similar to the one used to identify features for use in determining a patient's total SOFA score or SOFA component scores predicted at amounts of time into the future.

The model training system 236 may also be configured with a machine learning process to train and output one or more models, such as models 222, which are capable of predicting values for physiological parameters typically used for SOFA component score calculations for future points in time. For example, the machine learning process may be configured to generate models that upon input of features identified during a machine learning process, output predicted future values of physiological parameters used in the determination of SOFA component scores, for example, including but not limited to bilirubin levels, the Glasgow coma scale, platelet values, and/or creatinine levels.

The model training system 236 may also be configured with a machine learning process to train and output multiple models, such as models 222 that have been trained in the machine learning process based on non-overlapping or partially overlapping sets of features. In some implementations, the multiple models different sets of features can be implemented on the prediction server 216 to create a more robust system that includes an ensemble or collection of models. In such implementations, the prediction server may predict future total SOFA scores, future SOFA component scores, and current total and component SOFA scores more accurately in situations when certain physiological parameters used in a given model may be missing or incomplete.

FIG. 2C illustrates an example block diagram of a system 200c for predicting SOFA scores and SIRS scores using training models that are generated by multiple or different machine learning processes. The individual components and functionality of each component shown and described in FIG. 2C are identical to the components and respective functionality shown in FIG. 2A with the exception that the system 200c includes a server 246 configured with a SIRS model training system 248 and a SOFA model training system 250.

As shown in FIG. 2C, system 200c includes a server 246. The server 246 includes a SIRS model training system 248 and a SOFA model training system 250. The SIRS model training system 248 and a SOFA model training system 250 each generate respective training models (e.g., training models 230 and 242 as shown in FIGS. 2A and 2B) according to respective machine learning processes. For example, the SIRS model training system 248 receives patient data as training inputs in a machine learning process via network 214 and generates one or more trained SIRS score prediction models 249 capable of predicting a patient's SIRS score at specified amounts of time into the future. Similarly, the remote SOFA model training system 250 receives patient data as training inputs in a different machine learning process via network 214 and generates one or more trained SOFA score prediction models 251 capable of predicting a patient's total SOFA score of SOFA component scores at specified amounts of time into the future.

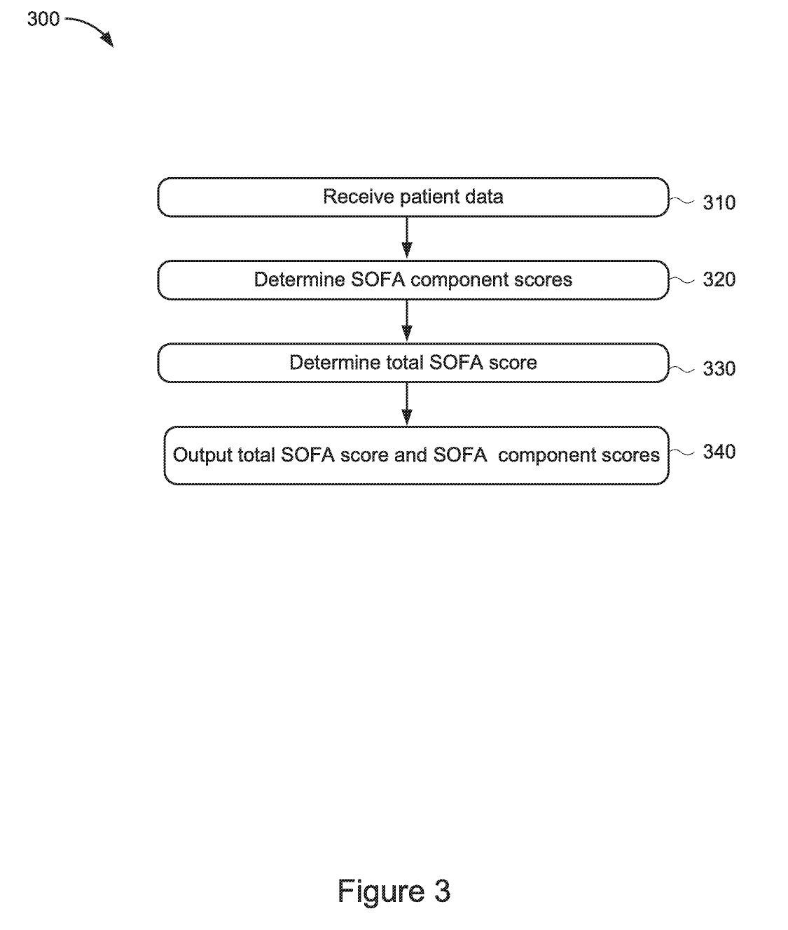

FIG. 3 illustrates an example method 300 for predicting SOFA scores using a SOFA score prediction model derived from a machine learning process performed by the example client 204 and servers 216, 234, and 246 of FIG. 2A-2C. The method 300 includes receiving patient data (stage 310). The method further includes determining the SOFA component scores (state 320) and determining the total SOFA score (stage 330). The method further includes outputting the total SOFA score and the SOFA component scores (stage 340).

At stage 310, the process 300 begins by receiving patient data at a server, such as server 216 shown in FIG. 2A. Patient data may be received from a variety of sources by a server which is configured with one or more trained SOFA score models that have been previously trained in a machine learning process to determine predicted SOFA scores for a patient at a specified amount of time into the future based on the received patient data. For example patient data may be stored on one or more computing devices, such as the large-format computing device 105 and the small-format computing device 110 shown in FIG. 1. In addition, patient data may be stored in a network-accessible database, such as the patient records database 115 as shown in FIG. 1. In some implementations, the database may be on a client device, such as client device 204 shown in FIG. 2A.

The received patient data may include patient identification data, standard clinical diagnostic data, as well other physiological data or clinical measurements that may seem unrelated to SOFA score prediction. The received patient data may include one or more data elements or feature that correspond to a specific clinical parameter or measurement obtained in a healthcare setting that may be used for the prediction of the SOFA scores. The patient data may include encounter data such as patient identifiers, the patient's date of birth, and the dates and times or admission or discharge from the hospital. The patient data may also include chart data identifying time stamps and numerical values for any treatments or actions taken by healthcare providers. The patient data may include laboratory data identifying time stamps and numerical values for the results of any diagnostic tests performed on the patient. The patient data may further include medication data identifying medication type, medication dosage, and time stamps for when the medication was administered to the patient.

The patient data, such as the prediction data associated with patient data 120 of FIG. 1, is received from the client 204 by a server, such as server 216 of FIG. 2A. The server 216 is configured with one or more trained SOFA score prediction models 232 that have been derived from a machine learning process and trained in the machine learning process to generate SOFA scores predicted for a patient a various amounts of time into the future. Each of the trained SOFA score prediction models 232 may generate a total SOFA score or a SOFA component score for a specific amount to time into the future. The trained SOFA score prediction model 232 processes the received patient data as model inputs in order to determine and output the predicted SOFA scores. The SOFA score prediction model 232 has been trained in a machine learning process to determine predicted total SOFA scores and SOFA component scores for a given patient at specified amounts of time into the future based on a wide variety of received patient data. The result of the machine learning process is a trained prediction model that is not limited to determining SOFA scores based solely on the requisite parameter inputs for SOFA score determination but is a trained prediction model that can utilize a broad range of patient data as model inputs to predict future SOFA scores and SOFA component scores.

At stage 320, the server 216 determines the SOFA component scores. The received patient data is processed using one or more trained SOFA score models, such as the trained SOFA score prediction models 232 shown in FIG. 2A to determine the total SOFA score and the SOFA component scores for patient at specified points of time into the future. There are six (6) different SOFA component scores, one for the hepatic, neurological, respiratory, coagulation, renal, and cardiovascular organ systems. Each SOFA component score can range from a minimum value of zero (0) to a maximum value of four (4). Each of 6 SOFA component scores can be determined by the trained SOFA score model using a variety of patient data as prediction inputs.

Based on processing the received patient data, the SOFA component scores for a patient can be predicted at a specified amount of time into the future. For example, each of the trained SOFA score prediction models 232 may output a patient's predicted SOFA component scores at 6, 12, 18 or 24 hours into the future. In some implementations, other models may be used to predict SOFA scores for other times into the future without departing from the scope of this disclosure.

At stage 330, the server 216 determines the total SOFA score. The total SOFA score can range from a minimum value of zero (0) to a maximum value of twenty-four (24). In some implementations, the total SOFA score may be determined by summing the values for each of the six different SOFA component scores. In other implementations, the total SOFA score may be determined by one or more total SOFA score prediction models. In this example, the received patient data is processed as inputs to the SOFA score prediction model to determine the total SOFA score. The machine learning process uses a machine learning algorithm to train and derive a training model, such as the trained SOFA score prediction model 232 shown in FIG. 2A that is capable of generating the total SOFA score based on received patient data that includes the same, more, less, and/or different features and feature values as compared to the features values in the patient data which specifically correspond to the model parameters necessary to compute each of the six different SOFA component scores.

Based on processing the received patient data the total SOFA score for a patient can be predicted at a specified amount of time into the future. For example, the trained SOFA score prediction model 232 may output a patient's predicted total SOFA scores at 6, 12, 18 and/or 24 hours into the future.

At stage 340, the server 216 outputs the total SOFA score and the SOFA component scores. In some implementations, the output total SOFA score and the SOFA component scores may be output to memory located on the server, for example memory 222 on server 216 as shown in FIG. 2A. In other implementations, the outputted total SOFA score and the SOFA component scores may be stored in a database, such as patient records database 115 shown in FIG. 1. In this example, the patient records database 115 may be configured on client 204 and/or on servers 216, 234, or 246.

In some implementations, the server 216 may output the total SOFA score and the SOFA component scores to client 204 shown in FIG. 2A. In some implementations, the client 204 may include a graphical user interface to display the output total SOFA score and the SOFA component scores. For example, application 210 on client 204 may include a graphical user interface to display the outputted total SOFA scores and the SOFA component scores. For example, the client 204 may be a monitor in the intensive care unit (ICU) of a hospital used to display patient data. The outputted total SOFA scores and the SOFA component scores may be displayed in a graphical user interface on the monitor to enable healthcare practitioners in the ICU to view patient's total SOFA scores and the SOFA component scores predicted for amount of time into the future. Displaying the predicted total SOFA scores and the SOFA component scores allows healthcare practitioners to prepare for and/or perform preventative therapeutic interventions, treatments or actions as appropriate for the patients' predicted SOFA scores. In other implementations, the server 216 output the total SOFA score and SOFA component scores to the client 204 and the client 204 may further store the outputted total SOFA scores and the SOFA component scores in memory 208. In some implementations, the server 216 output the total SOFA score and SOFA component scores to the client 204 and the client 204 may further output the total SOFA score and SOFA component scores to output device 202.

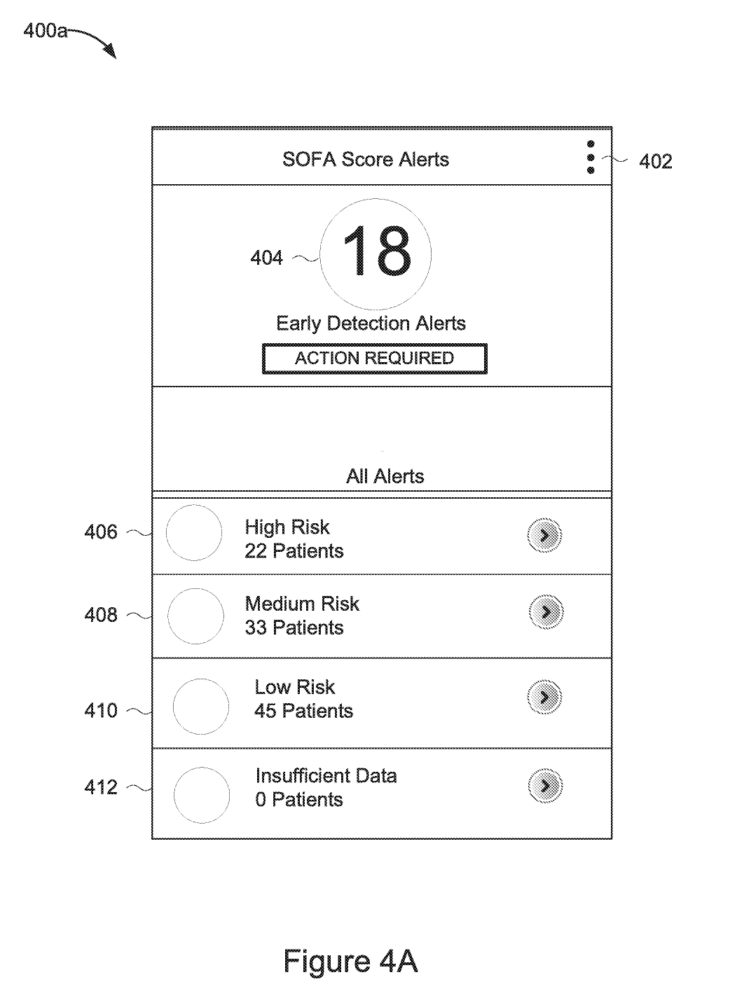

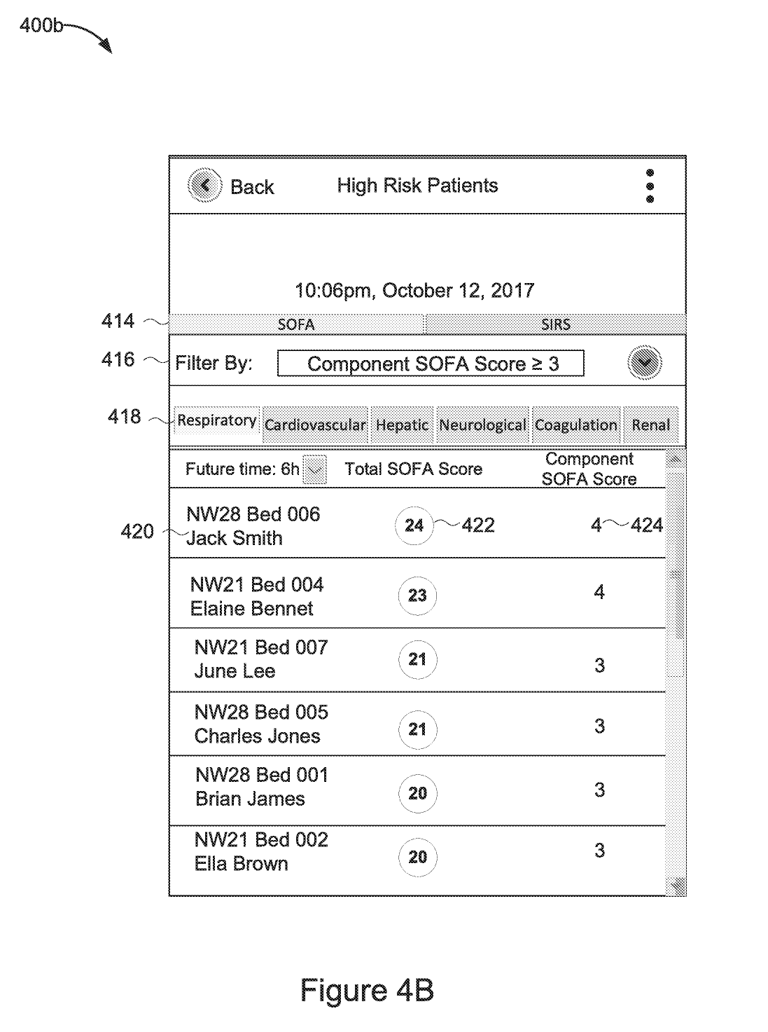

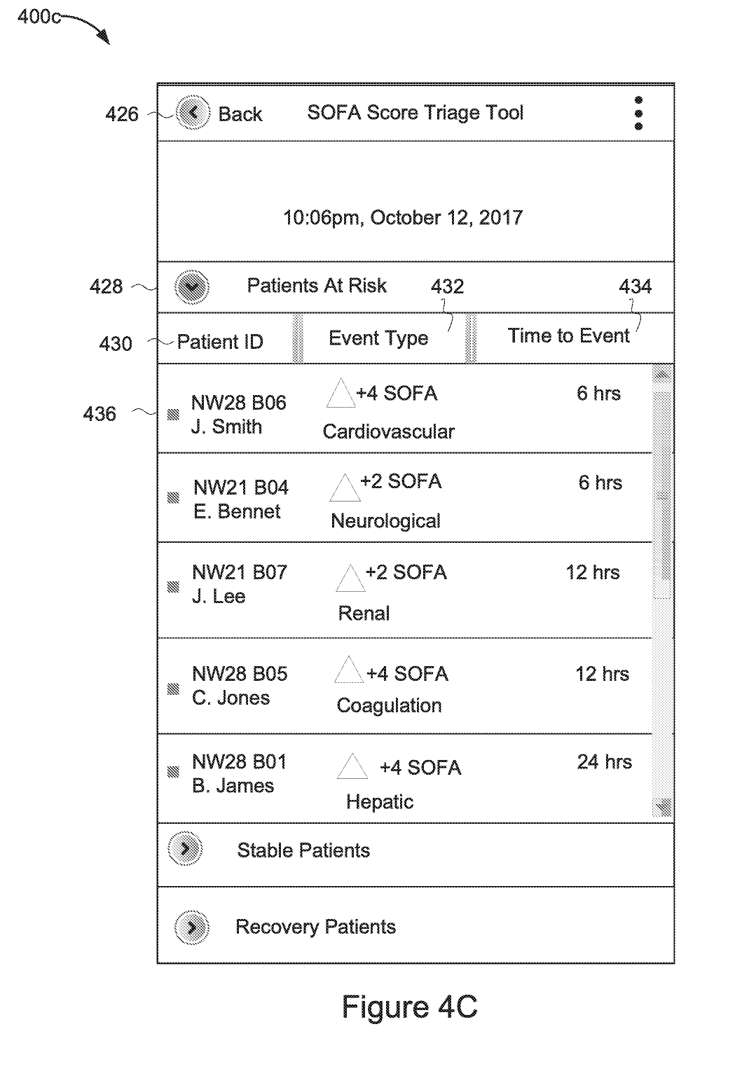

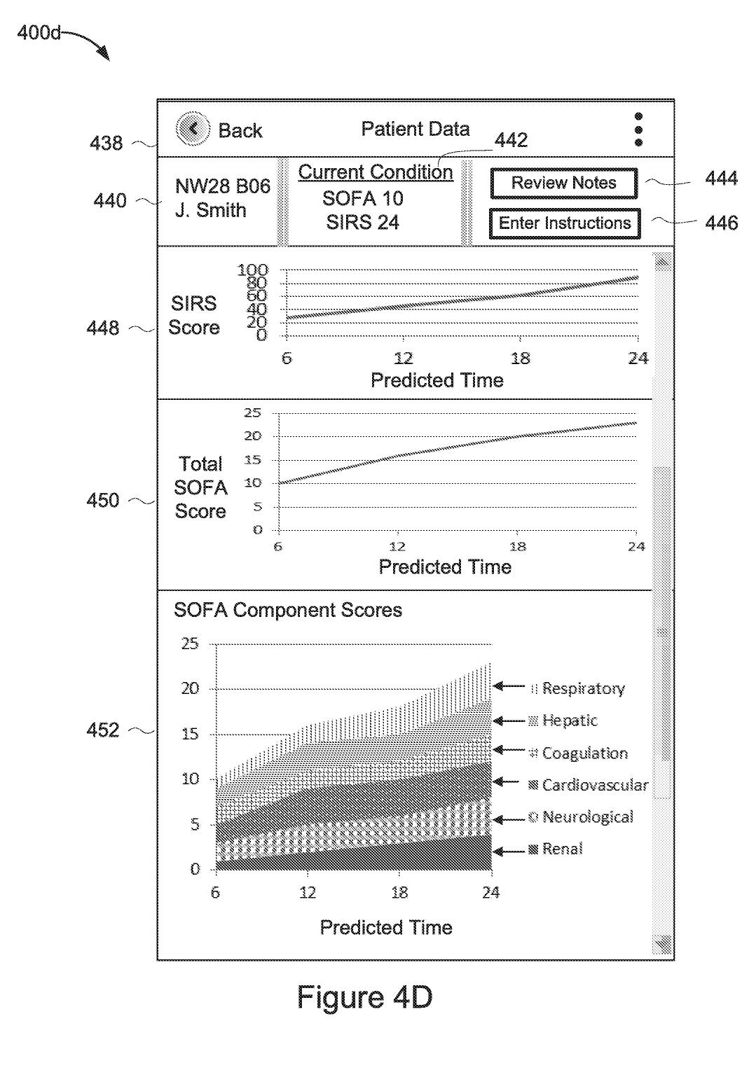

FIGS. 4A-4D illustrate example user interfaces for displaying and interacting with patients' predicted SOFA scores on small-format computing devices according to some implementations. The user interfaces shown in FIGS. 4A-4D allow a healthcare practitioner to receive predicted SOFA scores for one or more patients and take actions based on the received predicted SOFA scores. In some implementations, the small-format device displaying the user interfaces may be a tablet, smart phone, or other similar small-format computing device used to maintain, input, receive, display, and/or transmit patient data. In some implementations the small-format computing device may be a clinical diagnostic device configured with a display, such as an electrocardiogram (EKG), a non-invasive ventilator, or a hemodynamic monitoring system. The clinical diagnostic device may be further configured to display the predicted SOFA scores for a patient at various amounts of time into the future on a user interface.

FIG. 4A illustrates an example user interface 400a for displaying and interacting with patient's predicted SOFA scores on a small-format computing device. User interface 400a includes a system settings element 402, an alert total count indicator 404, a high risk patient count indicator 406, a medium risk patient count indicator 408, a low risk patient count indicator 410 and an insufficient data indicator 412.

As shown in FIG. 4A, the user interface 400a provides healthcare practitioners with a graphical display identifying predicted SOFA score alert data and patient risk categories. For example, the user interface 400a includes a system settings element 402, which is an interactive element for accessing system settings or configuration details. The user interface 400a also includes an alert total count indicator 404. The alert total count indicator 404 informs the healthcare practitioner of the number of patients requiring preventive therapeutic treatments at an amount of time in the future based on the predicted SOFA scores. The alert total count indicator 404 identifies the number of patients whose individual total SOFA score exceeds a threshold value. Additionally, or alternatively, the alert total count indicator 404 may also be configured to identify the number of patients for whom one or more SOFA component scores (of the 6 predicted SOFA component scores) exceeds a threshold value. For example, the alert total count indicator 404 shown in FIG. 4A indicates there are 18 patients for whom a total SOFA score or SOFA component score is predicted to exceed a predetermined threshold value at a specified time in the future, and that healthcare practitioners should review the individual patient's data to determine the appropriate next course of action for treatment. By selecting or clicking on the alert total count indicator 404, the user interface 400a may present to the healthcare practitioner a list of the 18 patients whose total SOFA score or a SOFA component score is predicted to exceed a predetermined threshold value at a specified amount of time into the future.

In some implementations, the alert total count indicator 404 may be configured to represent all alerts that have been generated irrespective of whether or not the patient's SOFA scores have exceeded a threshold value. For example, the alert total count indicator 404 could be configured to trigger an alert for any change in a patient's predicted total SOFA score or SOFA component scores. By selecting or clicking on the alert total count indicator 404, the user interface 400a may present to the healthcare practitioner a list of all patients whose predicted total SOFA score or any of the patient's one or more SOFA component scores have changed since the last determination of the patient's predicted SOFA scores. Using this displayed data, a team of healthcare practitioners may better manage treatment options and treatment delivery timing based on the predicted changes in the patient's predicted SOFA scores.

User interface 400a includes a high risk patient count indicator 406. The high risk patient count indicator 406 provides data to the healthcare practitioner about the number of patients whose predicted SOFA scores indicate that the patient is currently experiencing or is predicted to experience a change in SOFA scores that places the patient in a high risk category requiring immediate review of the patient's data for possibly urgent treatment. The assignment of a patient to the high risk category may be based on a patient's predicted SOFA score exceeding a user-configured threshold value or based on a user-configured amount of change identifying the magnitude by which one or more of the patient's predicted SOFA scores changes in one or more amounts of time into the future. In some implementations, the high risk patient count indicator 406 may be accompanied by a colored icon (such as red circle). In other implementations the high risk patient count indicator 406 may be accompanied by an animated icon (such as a flashing exclamation point). The high risk patient count indicator 406 may also include an interactive element, which when selected in the user interface will provide the healthcare practitioner with the list of patients determined to be in the high risk category based on their predicted SOFA scores. For example, as shown in user interface 400a, the high risk patient count indicator 406 includes an icon displaying a right pointing chevron within a circle, which when selected displays the list of patients in the high risk category in the user interface 400a.

User interface 400a includes a medium risk patient count indicator 408. The medium risk patient count indicator 408 provides data to the healthcare practitioner about the number of patients whose predicted SOFA scores indicate that the patient is currently experiencing or is predicted to experience a change in SOFA scores that places the patient in a medium risk category requiring vigilant monitoring and observation of the patient's data to prevent the need for more urgent treatment. The assignment of a patient to the medium risk category may be based on a change in a patient's predicted SOFA score exceeding a user-configured threshold value or based on a user-configured amount of change identifying the magnitude by which one or more of the patient's predicted SOFA scores changes at one or more amounts of time into the future. In some implementations, the medium risk patient count indicator 408 may be accompanied by a colored icon (such as yellow circle). In other implementations, the medium risk patient count indicator 408 may be accompanied by an animated icon. As described above in relation to the high risk patient count indicator 406, the medium risk patient count indicator 408 may also include an interactive element, which when selected in the user interface will provide the healthcare practitioner with the list of patients determined to be in the medium risk category based on their predicted SOFA scores.

User interface 400a includes a low risk patient count indicator 410. The low risk patient count indicator 410 provides data to the healthcare practitioner about the number of patients whose predicted SOFA scores indicate that the patient is currently experiencing or is predicted to experience a change in SOFA scores that places the patient in a low risk category requiring minimal and routine monitoring of the patient's data to prevent the need for further treatment. The assignment of a patient to the low risk category may be based on a change in a patient's predicted SOFA score exceeding a user-configured threshold value or based on a user-configured amount of change identifying the magnitude by which one or more of the patients predicted SOFA scores changes at one or more amounts of time into the future. In some implementations, the low risk patient count indicator 410 may be accompanied by a colored icon (such as green circle). In other implementations the low risk patient count indicator 410 may accompanied by be an animated icon. As described above in relation to the high risk patient count indicator 406, the low risk patient count indicator 410 may also include an interactive element, which when selected in the user interface will provide the healthcare practitioner with the list of patients determined to be in the low risk category based on their predicted SOFA scores.

User interface 400a includes an insufficient data indicator 412. The insufficient data indicator 412 provides data to the healthcare practitioner about the number of patients for whom there is not sufficient patient data available to predict SOFA scores. For example, patients who are newly admitted to the ICU may not have enough associated patient data to be used for predicting their SOFA scores at a specified amount of time into the future. As more data is generated for the patient, the predicted SOFA scores may be determined for the patient and the patient may be assigned to the low, medium or high risk categories based on the determined SOFA scores predicted at one or more amounts to time into the future. In some implementations, the insufficient data indicator 412 may be accompanied by a colored icon. In other implementations the insufficient data indicator 412 may be accompanied by an animated icon. As described above in relation to the high risk patient count indicator 406, the insufficient data indicator 412 may also include an interactive element, which when selected in the user interface will provide the healthcare practitioner with the list of patients determined to be in the insufficient data category.

FIG. 4B illustrates an example user interface 400b on a small-format computing device for displaying and interacting with patients who have been assigned to a particular risk category, for example the high risk category, based on the patient's predicted SOFA scores. The user interface 400b includes an interactive element to navigate back to the user interface 400a (e.g., shown as an icon displaying a left pointing chevron within a circle) as well as an interactive element for system settings or configuration details (e.g., shown as three vertical dots, which is identical to system settings element 402 described in relation to FIG. 4A). The user interface 400b also includes prediction score tabs 414, patient data filters 416, SOFA component score tabs 418, patient identification data 420, patient total SOFA score data 422, and patient SOFA component score data 424.

As shown in FIG. 4B, the user interface 400b provides healthcare practitioners with a graphical display identifying a list of patients who have been assigned to a particular risk category based on the patient's predicted SOFA scores. For example, the user interface 400b is displaying a list of patients who have been assigned to a high risk category. The user interface 400b includes prediction score tabs 414. The prediction score tabs 414 enable a healthcare practitioner to view SOFA score data or SIRS score data for the list of patients in the risk category. Based on selecting the SOFA prediction score tab, the user interface will display data related to the patient's SOFA scores. Similarly, based on selecting the SIRS prediction score tab, the user interface will display data related to the patient's SIRS score.

As further shown in FIG. 4B, the user interface 400b includes patient data filters 416. The patient data filters 416 enable a healthcare practitioner to filter the SOFA or SIRS score data by a list of predetermined thresholds. For example, as shown in user interface 400b, the healthcare practitioner has selected the prediction tab 414 designated for displaying predicted SOFA scores for the listed patients assigned to the high risk category. The healthcare practitioner may further select a patient data filter 416 to refine the list of patients based on the filter criteria. As shown in user interface 400b, the healthcare practitioner has selected to filter the list of all high risk patients such that the user interface displays only the high risk patients for whom at least one SOFA component score is greater than or equal to a value of three (3). Upon executing the specific filter command that the healthcare practitioner has selected, the user interface 400b will display the list of patients for whom at least one SOFA component score is greater than or equal to a value of three. The patient data filter 416 may include a variety of pre-configured or user-defined filter selection settings corresponding to range of possible values associated with each of the predicted SOFA scores and the predicted SIRS score. In some implementations, the patient data filter 416 may include a filter selection setting for applying no filter.

As further shown in FIG. 4B, the user interface 400b includes SOFA component score tabs 418. The SOFA component score tabs 418 enable a healthcare practitioner to view SOFA component score data for the list of patients. For example, as shown in user interface 400b, the healthcare practitioner has selected to display the SOFA component score tab 418 related to the respiratory organ system. Upon selection, the user interface 400b will display the respiratory SOFA component score data for each patient. In some implementations, the SOFA component score tab 418 may be used in conjunction with the patient data filter 416 and in other implementations, the patient data filter 416 may be set to apply no filter to the selected display of SOFA component scores corresponding to the selected SOFA component score tab 418.

As further shown in FIG. 4B, the user interface 400b includes patient identification data 420. The patient identification data 420 includes personal and administrative data for use by healthcare practitioners for determining the identity and location of a particular patient. For example, the patient identification data 420 may include, but is not limited to, the patient's name, the hospital ward in which the patient is being treated, and the bed number that the patient is occupying in the hospital ward. A wide variety of other personal and administrative data could also be presented as patient identification data 420. In some implementations, the display of the specific patient identification data 420 may be user-defined or may be pre-configured.

As further shown in FIG. 4B, the user interface 400b includes patient total SOFA score data 422. The patient total SOFA score data 422 identifies each patient's current or most recently determined total SOFA score predicted for a user-selectable amount of time into the future. The patient total SOFA score data 422 includes the value of the total SOFA score predicted for the time in the future corresponding to the user selection. For example, as shown in user interface 400b, the patient total SOFA score data 422 predicted for patient Jack Smith, for a user-selected time of 6 hours into the future, is 24. In some implementations, the patient total SOFA score data 422 may be displayed with an interactive element indicating the risk category that the particular patient has been assigned based on the patient's predicted SOFA scores.

As further shown in FIG. 4B, the user interface 400b includes patient SOFA component score data 424. The patient SOFA component score data 424 identifies each patient's current or most recently determined SOFA component score predicted for a user-selectable amount of time into the future. For example, the healthcare practitioner may wish to see a patient's SOFA component score data 424 predicted for a time 6 hours into the future. In some implementations, the patient's SOFA component score data 424 corresponds to the selected patient data filter 416 setting and the selected SOFA component score tab 418. In other implementations when no patient data filter 416 setting is applied, the user interface 400b displays the patient SOFA component score data 424 based on only on the SOFA component score tab 418 selected by the healthcare practitioner. In some implementations, the user interface 400b may include additional columns to display the patient's current total SOFA score or current SOFA component scores estimated for the present time. Similarly, if a user selected to view the SIRS prediction score tab 414, the user interface 400b may display the current SIRS score which have been estimated for the present time.

In some implementations, a user, such as a healthcare practitioner, may interact with the patient's SOFA component score data 424 shown in user interface 400b and the user interface will display the physiological parameters typically associated with calculating the particular SOFA component score predicted to occur at some amount of time into the future. For example, consider the user interface 400b shown FIG. 4B where a healthcare practitioner is viewing the SOFA score data for Jack Smith predicted at six hours into the future. Assume the healthcare practitioner has selected the “Respiratory” SOFA component score tab 418 as shown. The user interface displays Jack Smith's respiratory SOFA component score data, e.g., the respiratory SOFA component score 424 that is predicted to reach a value of “4” at a time of six hours into the future. In some implementations, the healthcare practitioner may interact with the SOFA component score data 424 by touching, clicking or selecting the displayed value, e.g., the “4” or the space or regions of the user interface surrounding the displayed value. In some implementations, the healthcare practitioner may interact with the SOFA component score data 424 by moving a pointer or mouse cursor over the displayed value, e.g., the “4”. Upon selecting or otherwise interacting with the SOFA component score “4”, the user interface 400b may display physiological parameter values that are associated with the predicted SOFA component. In some implementations, the predicted physiological parameter value is used in predicting the future SOFA component score. In some implementations, the future SOFA component score is predicted using a model that does not take into account the predicted physiological parameter value. For example, the user interface 400b, upon selecting or clicking the “4”, may display a data table that is superimposed atop the existing user interface 400b identifying the physiological parameters and parameter values that correspond to Jack Smith's respiratory SOFA component score, such as the measured ratio between Jack Smith's PAO2 (arterial partial pressure) and FIO2 (fraction of inspired oxygen). In some implementations, the user interface 400b, upon selecting or clicking the “4”, may transition to or display a new user interface (not shown) where the data identifying with the physiological parameters and parameter values that correspond to Jack Smith's respiratory SOFA component score are displayed.

In some implementations, the user interface 400b may be configured to further display SOFA score data for an individual patient by clicking or selecting interactive elements such as icons or links associated with any of the patient identification data 420, patient total SOFA score data 422, and patient SOFA component score data 424. The user interface displaying SOFA score data for an individual patient will be described in relation to FIG. 4D.

FIG. 4C illustrates an example user interface 400c on a small-format computing device for displaying and interacting with a list of patients requiring triage or possible treatment based on predicted changes in the patient's SOFA scores. By presenting the list of patients and their predicted event types, healthcare practitioners can better prepare treatment options and resources to provide preventative care to the listed patients. The user interface 400c includes interactive elements 426 to navigate back to user interfaces 400a or 400b as well as interactive elements for system settings or configuration details (e.g., shown as three vertical dots). The user interface 400c also includes one or more patient triage categories 428, patient identification data 430, triage event type data 432, event timing data 434, and individual patient triage data 436.

As shown in FIG. 4C, the user interface 400c displays a list of patients requiring triage or possible treatment based on predicted changes in the patient's SOFA scores. The user interface 400c includes one or more patient triage categories 428. The patient triage category 428 is assigned based on the type and severity of SOFA score events that are determined based on the patients' SOFA scores predicted at one or more amounts of time into the future. For example, as shown in user interface 400c, the patient triage categories 428 include “Patients At Risk,” “Stable Patients,” and “Recovery Patients”. Upon selecting the icon (e.g., shown as an icon displaying a downward pointing chevron within a circle) to display the patient triage category 428 “Patients At Risk,” the user interface 400c will display the list of patients who are predicted to experience some change in SOFA scores (or event) that is likely to require urgent triage or treatment by healthcare practitioners.

As further shown in FIG. 4C, the user interface 400c includes patient identification data 430. The patient identification data 430 is similar, but not limited to, the patient identification data 420 described in relation to FIG. 4B. User interface 400c may include additional or fewer patient identification data elements as required to accurately identify individual patients requiring triage or possible treatment based on predicted changes in the patient's SOFA scores.

As further shown in FIG. 4C, the user interface 400c includes triage event type data 432. The triage event type data 432 identifies the event associated with a specific change in a patient's SOFA scores (e.g., an event or a change event). The event associated with a specific change in a patient's SOFA scores may include an increase or a decrease in one of the patient's SOFA component scores or the patient's total SOFA score. Patients may be assigned to a particular patient triage category 428 based on the event type data 432. Healthcare practitioners may use the event type data 432 to rapidly determine triage and treatments plans for the patient who is experiencing the change in one or more SOFA scores. In some implementations, the triage event type data 432 may include symbols (such as a triangle, exclamation point or colored icon) alerting the healthcare practitioner that a patient has experienced event associated with one or more SOFA scores. In some implementations, the event type data 432 may include an indication of the magnitude of change in a particular SOFA score predicted at an amount of time in the future. In some implementations, the event type data 432 may include the specific organ system that corresponds to the SOFA components score for which the event is predicted to occur.

As shown in FIG. 4C, the user interface 400c includes event timing data 434. The event timing data 434 identifies the amount of time in the future that the predicted event or specific change in a patient's SOFA scores will occur. Healthcare practitioners may use the event timing data 434 to more accurately plan when to prepare and initiate treatments plans for patients who are predicted to experience a change in one or more SOFA scores at the specified amount of time into the future. In some implementations, the event timing data 434 may be used to trigger alerts in the user interface 400C or notifications, for example a text message that may be sent to a small-format computing device such as a mobile phone or tablet computing device to inform the healthcare practitioners of the predicted event. The alerts or notifications may include the event timing data 434, the triage event type data 432, and the patient identification data 430.

As further shown in FIG. 4C, the user interface 400c includes individual patient triage data 436. The individual patient triage data 436 includes the patient identification data 430, the event timing data 434, and the triage event type data 432 for a particular patient. For example, as shown in user interface 400c, the individual patient triage data 436 indicates that patient J. Smith, located in hospital ward NW28 in bed B06 will experience a triage event 6 hours into the future. The individual patient triage data 436 for patient J. Smith further indicates that the patient's total SOFA score will increase by 4 (e.g., for example from 20 to 24). The individual patient triage data 436 for patient J. Smith further indicates that the total SOFA score increase of 4 is attributed to a change in the SOFA component score associated with the cardiovascular organ system. In some implementations, the individual patient triage data 436 may display triage event type data 432 associated with events corresponding to changes in SOFA components scores. The user interface 400c displaying the patient identification data 430 also includes user interface controls (shown as a + symbol within a box) to view additional details associated with the individual patient's predicted SOFA scores as will be described in relation to FIG. 4D.

FIG. 4D illustrates an example user interface 400d on a small-format computing device displaying SOFA score data for an individual patient. Healthcare practitioners may interact with user interface 400d to review a patient's current condition, review a patient's predicted SOFA and SIRS scores, review the patient's treatment notes, and to enter treatment instructions for the patient. The user interface 400d includes an interactive element 438 to navigate back to user interfaces 400b or 400c as well as interactive elements for system settings or configuration details (e.g., shown as three vertical dots). The user interface 400d also includes patient identification data 440, current condition data 442, a review notes element 444, an enter instructions element 446, a SIRS score prediction graph 448, a total SOFA score prediction graph 450, and a SOFA component score graph 452.

As shown in FIG. 4D, the user interface 400d includes patient identification data 440. The patient identification data 440 is similar to the patient identification data 420 shown in FIG. 4B. User interface 400d may include additional or fewer patient identification data elements as required to accurately identify individual patients in the context displaying SOFA score data for an individual patient.

As further shown in FIG. 4D, the user interface 400d includes current condition data 442. The current condition data 442 indicates the patient's current total SOFA score or current SIRS score estimated for the present time. In some implementations, the current condition data 442 may identify the SOFA and SIRS scores that are determined based on manual input into a patient data records interface that is associated with user interface 400d (not shown).

As further shown in FIG. 4D, the user interface 400d includes a review notes element 444. The review notes element 444 is a graphical element in the user interface 400d that, when selected, displays the patient's medical charts, treatment notes, and/or any other configured data that has been linked to the review notes element to enable healthcare practitioners to view additional data pertaining to the patient's treatment in the hospital.

As further shown in FIG. 4D, the user interface 400d includes an enter instructions element 446. The enter instructions element 446 is a graphical element in the user interface 400d that, when selected, displays an interface for the healthcare practitioner to enter instructions about the patient's treatment or care. For example, upon reviewing a patient's predicted SOFA score data displayed on the user interface 400d, the healthcare practitioner may take action to treat or manage a predicted change event associated with an organ system corresponding to the predicted change event by selecting the enter instructions element 446 and entering the instructions. The healthcare practitioner may select the enter instructions element 446 in the user interface 400d to enter instructions such as to prescribe a medication at a specific dosage, to request an EKG, to instruct laboratory work to be performed, or to inform other healthcare professionals to prepare the patient for an urgently needed procedure such as intubating the patient, placing the patient on a ventilator, or initiating dialysis on the patient.

As further shown in FIG. 4D, the user interface 400d includes a SIRS score prediction graph 448. The SIRS score prediction graph 448 is graph displaying the patient's predicted SIRS score over a specified amount of time into the future. One skilled in the art will appreciate that a variety of graphs types or graph formats may be used to display the SIRS score data in a SIRS score prediction graph 448.

As further shown in FIG. 4D, the user interface 400d includes a total SOFA score prediction graph 450. The total SOFA score prediction graph 450 is graph displaying the patient's predicted total SOFA scores over a specified amount of time into the future. One skilled in the art will appreciate that a variety of graphs types or graph formats may be used to display the total SOFA score data in a total SOFA score prediction graph 450.

As further shown in FIG. 4D, the user interface 400d includes a SOFA component score prediction graph 452. The SOFA component score prediction graph 452 is graph displaying the patient's predicted SOFA component scores over a specified amount of time into the future. In some implementations, the SOFA component score graph 452 may be a plot of all six of a patient's SOFA component scores over one or more specified amounts of time into the future. In other implementations, the SOFA component score graph 452 may be a plot of a single SOFA component score over one or more specified amounts of time into the future. By plotting the SOFA component scores predicted for a patient over one or more specified amounts of time into the future, healthcare practitioners can gain insight into the future condition of the specific organ system that is associated with a particular SOFA component score. The insight gained by viewing plots of the SOFA component scores will assist healthcare practitioners to prepare preventative treatments in advance that are directed at the specific organ systems for which the predicted SOFA component scores correspond. One skilled in the art will appreciate that a variety of graphs types or graph formats may be used to display the SOFA component score data in a SOFA component score prediction graph 452.

In some implementations, a user, such as a healthcare practitioner, may interact with elements of the patient's SOFA component score prediction graph 452 and the user interface 400b will display the physiological parameters typically associated with calculating the particular SOFA component score predicted to occur at some amount of time into the future. For example, consider the user interface 400d shown FIG. 4D where a healthcare practitioner is viewing the SOFA score data for Jack Smith. The user interface 400d displays Jack Smith's SOFA component score data in a graph, such as the SOFA component score prediction graph 452. The graph displays the predicted SOFA component scores that are respectively associated with each SOFA component score organ system as a function of 6, 12, 18, and 24 hours into the future. In some implementations, the healthcare practitioner may interact with the SOFA component score prediction graph 452 by touching, clicking or selecting an individual SOFA component score organ system identified in the legend (as shown on the left of the SOFA component score prediction graph 452) or clicking or selecting a data point located on or within the graph that corresponds to a specific SOFA organ system component score. In some implementations, the healthcare practitioner may interact with the SOFA component score prediction graph 452 by moving a pointer or mouse cursor over a particular SOFA component score organ system identified in the legend (as shown on the left of the SOFA component score prediction graph 452) or by moving a pointer or mouse cursor over a data point located on or within the graph that corresponds to a specific SOFA component score organ system. Upon selecting or otherwise interacting with the data or graphical elements of the SOFA component score prediction graph 452, the user interface 400d may display physiological parameter values that are associated with the predicted SOFA component score. In some implementations, the predicted physiological parameter value is used in predicting the future SOFA component score. In some implementations, the future SOFA component score is predicted using a model that does not take into account the predicted physiological parameter value. For example, the user interface 400d, upon selecting or clicking the “Respiratory” legend label, may display a data table that is superimposed atop the existing user interface 400d identifying the physiological parameters and parameter values that correspond to Jack Smith's respiratory SOFA component score, such as the measured ratio between Jack Smith's PAO2 (arterial partial pressure) and FIO2 (fraction of inspired oxygen). In some implementations, the user interface 400d, upon selecting or clicking the data point on the graph that is located within graph area corresponding to the respiratory SOFA component score, may transition to or display a new user interface (not shown) where the data identifying with the physiological parameters and parameter values that correspond to Jack Smith's respiratory SOFA component score are displayed.

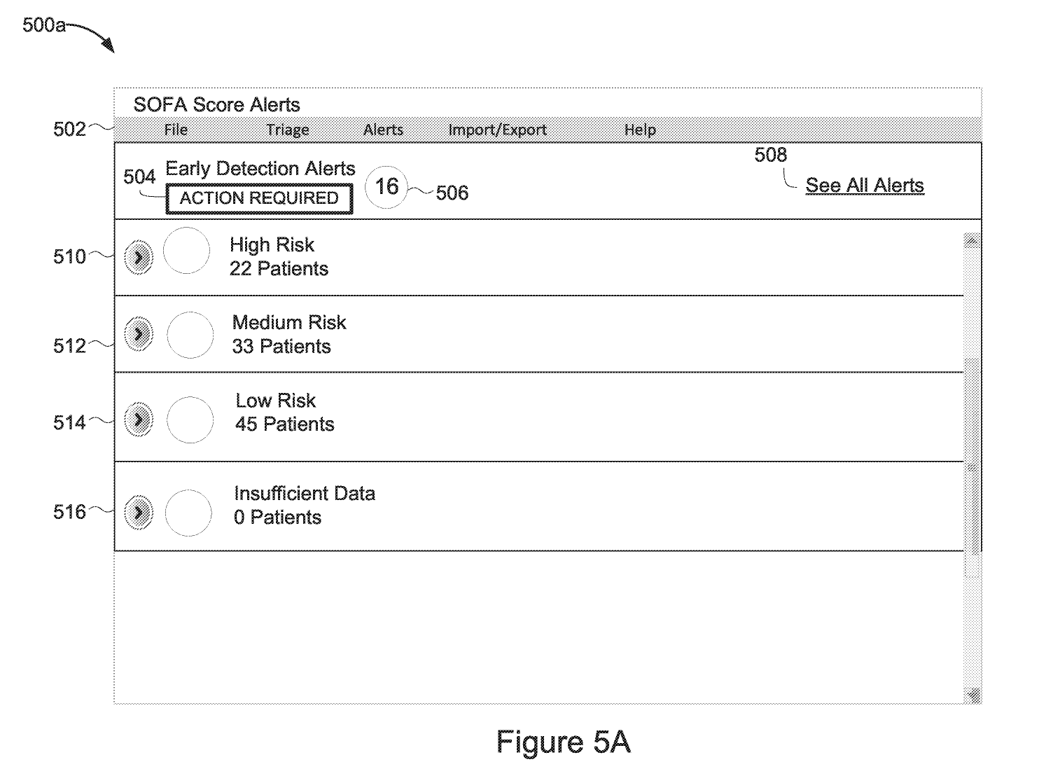

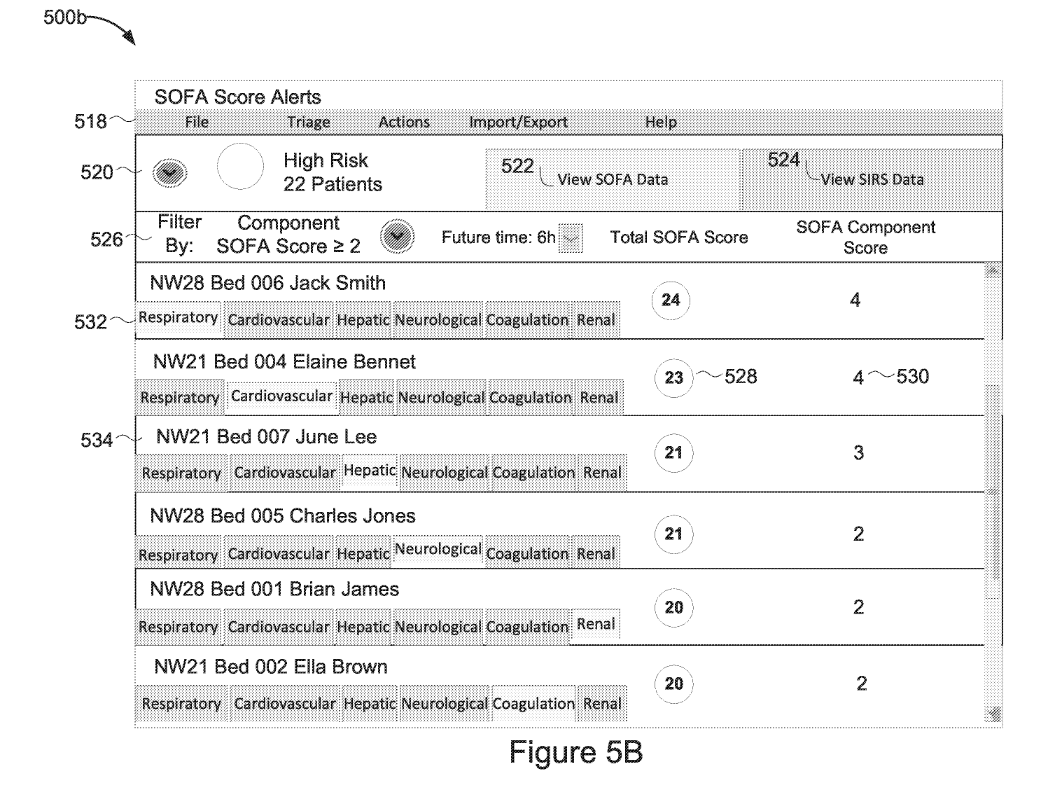

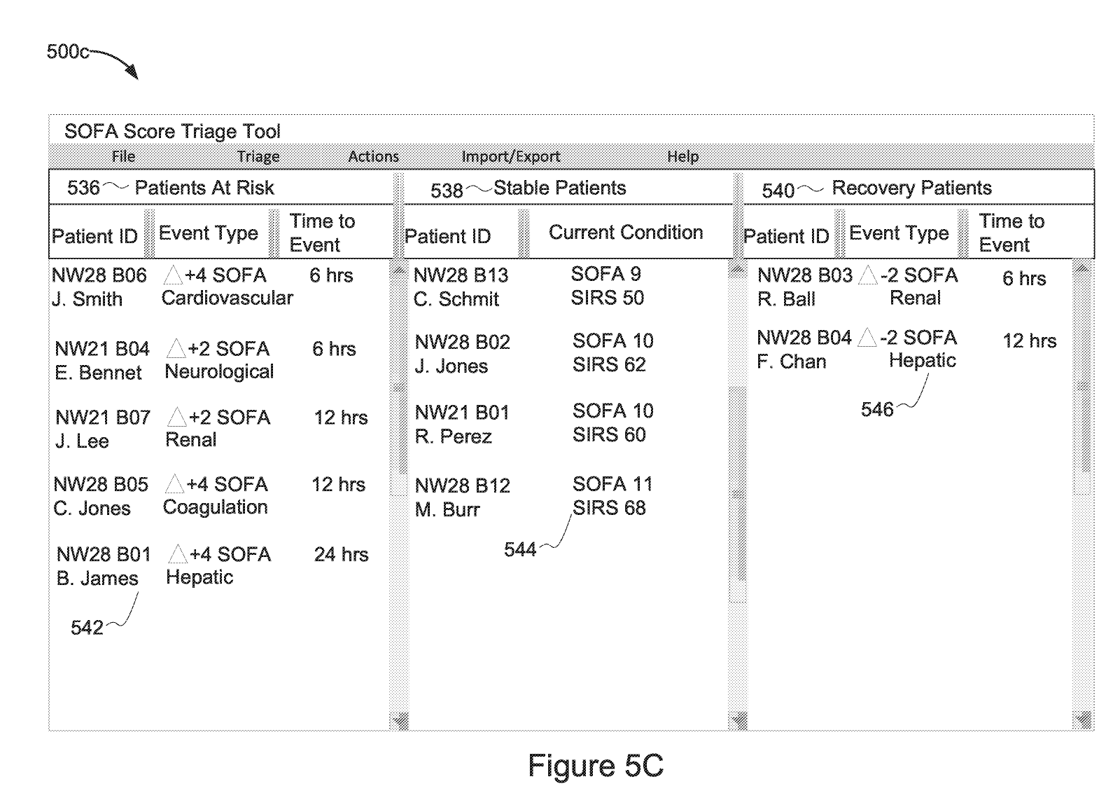

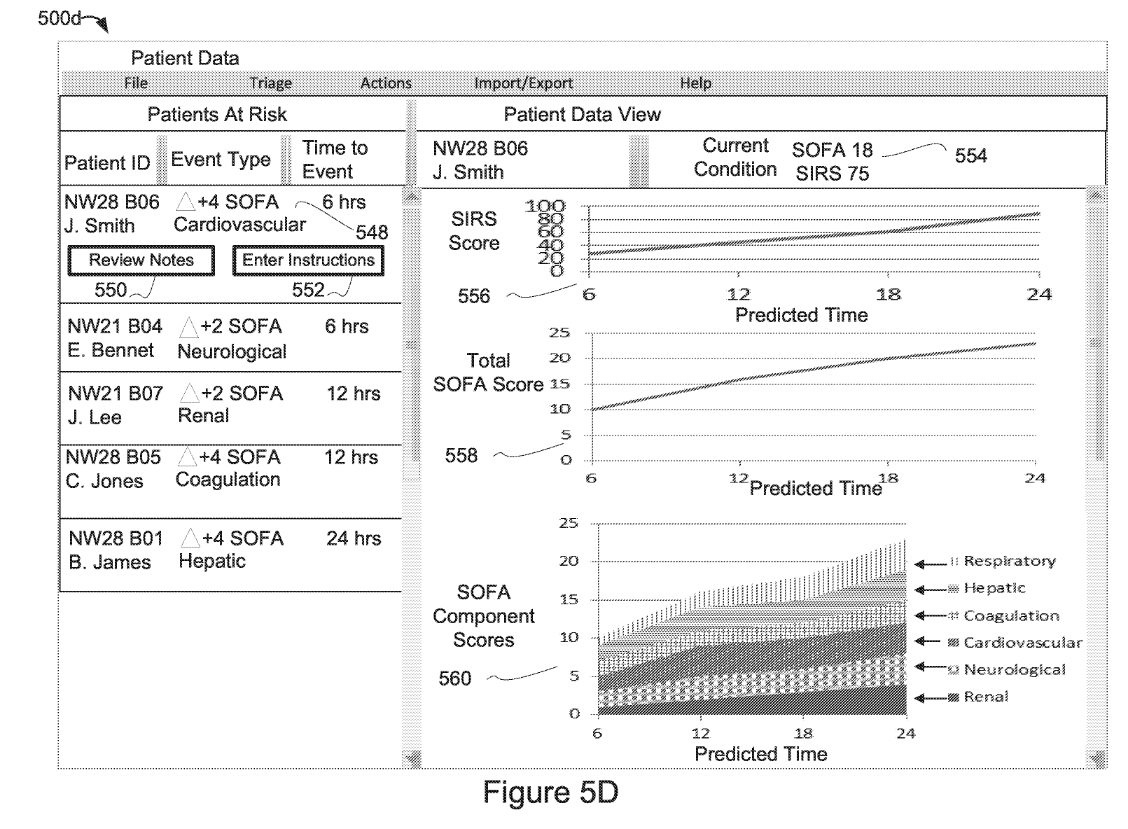

FIGS. 5A-5D illustrate example user interfaces for displaying and interacting with patients' predicted SOFA scores on large-format computing devices according to some implementations. In broad overview, FIGS. 5A-5D include similar components providing similar functionality as the components and functionality described in FIGS. 4A-4D pertaining to small-format computing devices. The user interfaces shown in FIGS. 5A-5D allow a healthcare practitioner to receive predicted SOFA scores for one or more patients and take actions based on the received predicted SOFA scores. In some implementations, the large-format computing device displaying the user interfaces may be a large-format computer, a computing terminal with a display, or other similar non-small-format computing devices used to maintain, input, receive, display, and/or transmit patient data. In some implementations the large-format computing device may be a clinical diagnostic device configured with a display, such as an electrocardiogram (EKG), a non-invasive ventilator, or a hemodynamic monitoring system. The clinical diagnostic device may receive inputs of patient data and transmit patient data that are specifically related to a particular patient data feature used to determine predicted SOFA scores. The clinical diagnostic device may be further configured to display the predicted SOFA scores for a patient at a specified amount of time into the future in the user interfaces.

FIG. 5A illustrates an example user interface 500a for displaying and interacting with patient's predicted SOFA scores on a large-format computing device. User interface 500a includes a menu 502, an action required indicator 504, an alert total indicator 506, an all alerts indicator 508, a high risk patient count indicator 510, a medium risk patient count indicator 512, a low risk patient count indicator 514 and an insufficient data indicator 516.

As shown in FIG. 5A, the user interface 500a provides healthcare practitioners with a graphical display identifying predicted SOFA score alert data and patient risk categories in a large-format computing environment. For example, user interface 500a includes a menu 502. The menu 502 is similar to commonly used large-format application menus and provides functionality for a variety of tasks to be performed in the application configured on the large-format computing device. For example, menu 502 includes a File sub-menu, a Triage sub-menu, an Alerts sub-menu, an Import/Export sub-menu, and a Help sub-menu. Each sub-menu includes a number of sub-menu selections that, when executed, perform various operations or tasks related to the sub-menu functionality. For example, the Triage sub-menu may include sub-menu selections to assist healthcare practitioners in the triage of patients whose predicted SOFA scores are generating alerts or notifications based on exceeding a threshold value. In another example, the Import/Export menu may include sub-menu selections to enable a healthcare practitioner to export the predicted SOFA score history that was maintained during a patient's stay in the hospital so that the predicted SOFA score history can be sent to a specialist for further evaluation of the patient. In addition, for example, the Alerts sub-menu item may provide healthcare practitioners with a consolidated list of all alerts that have been generated within a recent period of time based on one or more changes in the predicted SOFA component scores for patients in a particular risk category.

As further shown in FIG. 5A, user interface 500a includes an action required indicator 504. The action required indicator 504 informs the healthcare practitioner that there are a number of alerts requiring immediate attention. Various interactive elements can be used to draw the attention of the healthcare practitioner to the action required indicator 504. For example the action required indicator 504 may flash or be change colors when the number of alerts reaches a particular value. In some implementations, the action required indicator 504 may include animated text or be configured to emit an audible sound when the number of alerts requiring action exceeds some value.

As shown in FIG. 5A, the user interface 500a includes an alert total indicator 506, similar to the alert total count indicator 404 shown in FIG. 4A. The alert total indicator 506 identifies the number of patients requiring preventive therapeutic treatments based on the SOFA scores predicted for one or more amounts of time into the future. In some implementations, the alert total indicator 506 identifies the number of patients whose individual total SOFA score exceeds a threshold value. Additionally, or alternatively, the alert total indicator 506 may also be configured to identify the number of patients for whom one or more SOFA component scores (of the 6 predicted SOFA component scores) exceeds a threshold value. For example, alert total indicator 506 indicates there are 16 patients for whom a total SOFA score and/or one or more SOFA component scores are predicted to exceed a predetermined threshold value and that healthcare practitioners should review the individual patient's data to determine the appropriate next course of action for treatment. By selecting or clicking on the alert total indicator 506, the user interface 500a may present to the healthcare practitioner a list of the 16 patients whose total SOFA score and/or one or more SOFA component scores are predicted to exceed a predetermined threshold value at a specified amount of time into the future.

User interface 500a also includes an all alerts indicator 508, similar to the all alerts indicator 404 shown in FIG. 4A. The all alerts indicator 508 provides an interactive element for the healthcare practitioner to view all alerts that have been generated irrespective of whether or not the patient's SOFA scores have exceeded a threshold value. For example, the all alerts indicator 508 could be configured to record an alert for any change in a patient's predicted total SOFA score or SOFA component scores. By selecting or clicking on the alert total indicator 508, the user interface 500a may present to the healthcare practitioner a list of all patients whose predicted total SOFA score or SOFA component score has changed since the determination of the patient's predicted SOFA scores. Using this displayed data, a team of healthcare practitioners may better manage treatment options, treatment delivery timing and allocation of resources based on the predicted changes in the patient's predicted SOFA scores.

User interface 500a includes a high risk patient count indicator 510. The high risk patient count indicator 510 provides data to the healthcare practitioner about the number of patients whose predicted SOFA scores indicate that the patient is currently experiencing or is predicted to experience a change in SOFA scores that places the patient in a high risk category requiring immediate review of the patient's data for possibly urgent treatment. The assignment of a patient to the high risk category may be based on a patient's predicted SOFA score exceeding a user-configured threshold value or based on a user-configured amount of change identifying the magnitude by which one or more of the patient's predicted SOFA scores changes in one or more amounts of time into the future. In some implementations, the high risk indicator 510 may be accompanied by a colored icon (such as red circle). In other implementations the high risk indicator 510 may be accompanied by an animated icon (such as a flashing exclamation point). The high risk patient count indicator 510 may also include an interactive element (for example, the right pointing chevron within a circle shown in FIG. 5A), which when selected in the user interface will provide the healthcare practitioner with the list of patients determined to be in the high risk category based on their predicted SOFA scores.

User interface 500a includes a medium risk patient count indicator 512. The medium risk patient count indicator 512 provides data to the healthcare practitioner about the number of patients whose predicted SOFA scores indicate that the patient is currently experiencing or is predicted to experience a change in SOFA scores that places the patient in a medium risk category requiring vigilant monitoring and observation of the patient's data to prevent the need for more urgent treatment. The assignment of a patient to the medium risk category may be based on a change in a patient's predicted SOFA score exceeding a user-configured threshold value or based on a user-configured amount of change identifying the magnitude by which one or more of the patient's predicted SOFA scores changes at one or more amounts of time into the future. In some implementations, the medium risk indicator 512 may be accompanied by a colored icon (such as yellow circle). In other implementations, the medium risk indicator 512 may be accompanied by an animated icon such. The medium risk patient count indicator 512 may also include an interactive element (for example, the right pointing chevron within a circle shown in FIG. 5A), which when selected in the user interface will provide the healthcare practitioner with the list of patients determined to be in the medium risk category based on their predicted SOFA scores.

User interface 500a includes a low risk patient count indicator 514. The low risk patient count indicator 514 provides data to the healthcare practitioner about the number of patients whose predicted SOFA scores indicate that the patient is currently experiencing or is predicted to experience a change in SOFA scores that places the patient in a low risk category requiring minimal and routine monitoring of the patient's data to prevent the need for further treatment. The assignment of a patient to the low risk category may be based on a change in a patient's predicted SOFA score exceeding a user-configured threshold value or based on a user-configured amount of change identifying the magnitude by which one or more of the patient's predicted SOFA scores changes at one or more amounts of time into the future. In some implementations, the low risk indicator 514 may be accompanied by a colored icon (such as green circle). In other implementations the low risk indicator 514 may be accompanied by an animated icon. The low risk patient count indicator 514 may also include an interactive element (for example, the right pointing chevron within a circle shown in FIG. 5A), which when selected in the user interface will provide the healthcare practitioner with the list of patients determined to be in the low risk category based on their predicted SOFA scores.

User interface 500a includes an insufficient data indicator 516. The insufficient data indicator 516 provides data to the healthcare practitioner about the number of patients for whom there is not sufficient patient data available to predict SOFA scores. For example, patients who are newly admitted to the ICU may not have enough associated patient data to be used for predicting their SOFA scores at a specified amount of time into the future. As more data is generated for the patient, the predicted SOFA scores may be determined for the patient and the patient may be assigned to the low, medium or high risk categories based on the determined SOFA scores predicted at one or more amounts to time into the future. In some implementations, the insufficient data indicator 516 may be accompanied by a colored icon. In other implementations the insufficient data indicator 516 may be accompanied by an animated icon. The insufficient data indicator 516 may also include an interactive element (for example, the right pointing chevron within a circle shown in FIG. 5A), which when selected in the user interface will provide the healthcare practitioner with the list of patients determined to be in the insufficient data category.

FIG. 5B illustrates an example user interface 500b on a large-format computing device for displaying and interacting with patients who have been assigned to a particular risk category, for example the high risk category, based on the patient's predicted SOFA scores. The user interface 500b includes a menu 518, similar to menu 502 described in relation to FIG. 5A. The user interface 500b includes a patient category expansion element 520, which is an interactive element that expands or collapses the list of patients who have been assigned to a particular patient category, such as the High Risk patient category shown in FIG. 5B including 22 patients. The user interface 500b also includes a view SOFA data tab 522, a view SIRS data tab 524, a patient data filters 526, patient total SOFA score data 528, patient SOFA component score data 530, SOFA component score tabs 532, and patient identification data 534.

As shown in FIG. 5B, the user interface 500b provides healthcare practitioners with a graphical display identifying a list of patients who have been assigned to a particular risk category based on the patient's predicted SOFA scores. The user interface 500b includes a view SOFA data tab 522 and a view SIRS data tab 524. The view SOFA data tab 522 and the view SIRS data tab 524 enable a healthcare practitioner to view the SOFA score data and SIRS score data, respectively, for the list of patients in the risk category. Based on selecting either the view SOFA data tab 522 or the view SIRS data tab 524, the user interface will display data related to the patient's SOFA scores or display data related to the patient's SIRS score.

As further shown in FIG. 5B, the user interface 500b includes patient data filters 526, similar to patient data filters 416 shown in FIG. 4B. The patient data filters 526 enable a healthcare practitioner to filter the SOFA or SIRS score data by a list of predetermined thresholds. For example, as shown in user interface 500b, the healthcare practitioner has selected the view SOFA data tab 522 designated for displaying predicted SOFA scores for the listed patients assigned to the high risk category. The healthcare practitioner may further select a patient data filter 526 to refine the list of patients based on the filter criteria. As shown in user interface 500b, the healthcare practitioner has selected to filter the list of all high risk patients such that the user interface displays only the high risk patients for whom at least one SOFA component score is greater than or equal to a value of two (2). Upon executing the specific filter command that the healthcare practitioner has selected, the user interface 500b will display the list of patients for whom at least one SOFA component score is greater than or equal to a value of two. The patient data filter 526 may include a variety of pre-configured or user-defined filter selection settings corresponding to range of possible values associated with each of the predicted SOFA scores and the predicted SIRS score. In some implementations, the patient data filter 526 may include a filter selection setting for applying no filter.

As further shown in FIG. 5B, the user interface 500b includes patient total SOFA score data 528. The patient total SOFA score data 528 identifies each patient's current or most recently determined total SOFA score predicted for a user-selectable amount of time into the future. The patient total SOFA score data 528 includes the value of the total SOFA score predicted for the user-selected time in the future. For example, as shown in user interface 500b, the patient total SOFA score data 528 predicted for patient Elaine Bennet, for a user-selected time of 6 hours into the future, is 23. In some implementations, the patient total SOFA score data 528 may be displayed with an interactive element indicating the risk category that the particular patient has been assigned based on the patient's predicted SOFA scores.

As further shown in FIG. 5B, the user interface 500b includes patient SOFA component score data 530. The patient SOFA component score data 530 identifies each patient's current or most recently determined SOFA component score predicted for a user-selectable amount of time into the future. For example, the healthcare practitioner may wish to see a patient's SOFA component score data 530 predicted for a time 6 hours into the future. In some implementations, the patient's SOFA component score data 530 corresponds to the selected patient data filter 526 setting and the selected view SOFA component score tab 532. The SOFA component score tabs 532 enable a healthcare practitioner to view SOFA component score data for the list of patients. For example, as shown in user interface 500b, the healthcare practitioner has selected to display the SOFA component score tab 532 for patient Jack Smith that is related to the respiratory organ system. Upon selection, the user interface 500b will display the respiratory SOFA component score data for the particular patient. In some implementations, the SOFA component score tab 532 may be used in conjunction with the patient data filter 526 and in other implementations, the patient data filter 526 may be set to apply no filter to the selected display of SOFA component scores corresponding to the selected SOFA component score tab 526. In some implementations when no patient data filter 526 setting is applied, the user interface 500b displays the patient SOFA component score data 530 based on only on the SOFA component score tab 532 selected by the healthcare practitioner.

In some implementations, a user, as a healthcare practitioner, may interact with the patient's SOFA component score data 530 shown in user interface 400b and the user interface will display the physiological parameters typically associated with calculating the particular SOFA component score predicted to occur at some amount of time into the future. For example, consider the user interface 500b shown FIG. 5B where a healthcare practitioner is viewing the SOFA score data for Elaine Bennet predicted for six hours into the future. Assume the healthcare practitioner has selected the “Cardiovascular” SOFA component score tab 532 as shown. The user interface 500d displays Elaine Bennet's cardiovascular SOFA component score data, e.g., the cardiovascular SOFA component score 530 that is predicted to reach a value of “4” at a time of six hours into the future. In some implementations, the healthcare practitioner may further interact with the SOFA component score data 530 by clicking or selecting the displayed value, e.g., the “4” or the space or regions of the user interface surrounding the displayed value. In some implementations, the healthcare practitioner may interact with the SOFA component score data 530 by moving a pointer or mouse cursor over the displayed value, e.g., the “4”. Upon selecting or otherwise interacting with the SOFA component score “4”, the user interface 500b may display physiological parameter values that are associated with the predicted SOFA component. In some implementations, the predicted physiological parameter value is used in predicting the future SOFA component score. In some implementations, the future SOFA component score is predicted using a model that does not take into account the predicted physiological parameter value. For example, the user interface 500b, upon selecting or clicking the “4”, may display a data table that is superimposed atop the existing user interface 500b identifying the physiological parameters and parameter values that correspond to Elaine Bennet's cardiovascular SOFA component, such as Elaine Bennet's MAP (mean arterial pressure) and/or her administered vasopressor dosage information. In some implementations, the user interface 500b, upon selecting or clicking the “4”, may transition to or display a new user interface (not shown) where the data identifying with the physiological parameters and parameter values that correspond to Elaine Bennet's cardiovascular SOFA component are displayed.

As further shown in FIG. 5B, the user interface 500b includes patient identification data 534. The patient identification data 534 includes personal and administrative data for use by healthcare practitioners for determining the identity and location of a particular patient. For example, the patient identification data 534 may include, but is not limited to, the patient's name, the hospital ward in which the patient is being treated, the bed number that the patient is occupying in the hospital ward. A wide variety of other personal and administrative data could also be presented as patient identification data 534. In some implementations, the display of the specific patient identification data 534 may be user-defined or may be pre-configured.

In some implementations, the user interface 500b may be configured to further display SOFA score data for an individual patient by clicking or selecting interactive elements such as icons or links associated with any of the patient identification data 534, the patient total SOFA scores, and the patient SOFA component score. The user interface displaying SOFA score data for an individual patient will be described in relation to FIG. 5D.

FIG. 5C illustrates an example user interface 500c on a large-format computing device for displaying and interacting with a list of patients requiring triage or possible treatment based on predicted changes in the patient's SOFA scores. By presenting the list of patients and their predicted event types, healthcare practitioners can better prepare treatment options and resources to provide preventative care to the listed patients. The user interface 500c includes a display of patients in three triage categories: a patients-at-risk triage category 536, a stable-patients triage category 538, and a recovery-patients triage category 540. The user interface 500c also includes patient triage data 542, 544, and 546 for patients assigned to each respective triage category. Patients are assigned to a particular triage category based on the type and severity of SOFA score events that are determined based on the patient's SOFA scores predicted at one or more amounts of time into the future. Patients assigned to the patients at risk triage category 536 may include patients who are predicted to experience some change in SOFA scores (or event) that is likely to require urgent triage or treatment by healthcare practitioners at some amount of time in the future based on the patient's predicted SOFA scores. Patients assigned to the stable-patients triage category 538 may include patients whose predicted SOFA scores are stable over the predicted amounts of time into the future and do not indicate fluctuations or events likely to require more urgent triage or treatment by healthcare practitioners. Patients assigned to the recovery-patients triage category 540 may include patients whose predicted SOFA scores are declining which is an indication that their medical conditions may be improving. Accordingly, these patients may require less priority in triage situations compared to patients in the aforementioned triage categories.

As shown in FIG. 5C, the user interface 500c includes patient triage data, such as patient triage data 542, 544, and 546. The patient triage data displayed may be specific to the triage category for which a particular patient is assigned. For example, as shown in user interface 500c, the patient triage data 542 includes the patient identification data, event type data, and event timing data for a particular patient. For example, the individual patient triage data 542 for patient B. James indicates he/she is located in bed B01 of hospital ward NW28. The individual patient triage data 542 for patient B. James also indicates he/she will experience a triage event 24 hours into the future. The event timing data identify the amount of time in the future that the predicted event or specific change in a patient's SOFA scores will occur and may be used by healthcare practitioners to more accurately plan when to prepare and initiate treatments plans for patients who are predicted to experience a change in one or more SOFA scores at the specified amount of time into the future. In some implementations, the event timing data may be used to trigger alerts or notifications to inform the healthcare practitioners of the predicted event. The alerts or notifications may include the event timing data, the triage event type data, and the patient identification data. Referring back to the individual patient triage data for patient B. James, the patient triage data 542 further indicates that the patient's total SOFA score will increase by 4 (e.g., for example from 20 to 24). The individual patient triage data 542 for patient B. James further indicates that the total SOFA score increase of 4 is attributed to a change in the SOFA component score associated with the hepatic organ system.

As further shown in FIG. 5C, the user interface 500c displays different elements of patient data for patients assigned to the stable-patients triage category 538. For example, the patient triage data 544 for M. Burr includes the current SOFA score and the current SIRS score. The current condition data for includes the patient's most recently determined total SOFA score and SIRS score that are predicted to occur at the next specified time. In some implementations, the current condition data may identify the SOFA and SIRS score prediction values based on a specified time in the past. In other implementations, the current condition data may identify the SOFA and SIRS scores that are determined based on manual input into a patient data records interface that is associated with user interface 400d (not shown).

As shown in FIG. 5C, the patient triage data 546 for F. Chan who is assigned to the recovery-patients triage category 540 includes data indicating improving SOFA scores such as negative or declining SOFA score value changes predicated at amounts of time into the future. As described previously, in some implementations, the triage event type data may include symbols (such as a triangle, exclamation point or colored icon) alerting the healthcare practitioner that a patient has experienced an event associated with one or more SOFA scores. In other implementations, the triage event type data may include an indication of the magnitude of change in a particular SOFA score predicted at an amount of time in the future. In some implementations, the triage event type data may include the specific organ system that corresponds to the SOFA components score for which the event is predicted to occur.

FIG. 5D illustrates an example user interface 500d on a large-format computing device displaying SOFA score data for an individual patient. Healthcare practitioners may interact with user interface 500d to review a patients current condition, review a patient's predicted SOFA and SIRS scores, review a patient's treatment notes, and to enter treatment instructions for the patient. The user interface 500d includes patient data 548, a review notes element 550, an enter instructions element 552, current condition data 554, a SIRS score prediction graph 556, a total SOFA score prediction graph 558, and a SOFA component score graph 560.

As shown in FIG. 5D, the user interface 500d includes patient identification data 548. The patient identification data 548 is similar to the patient identification data 542 shown in FIG. 5C. User interface 500d may include additional or fewer patient identification data elements as required to accurately identify individual patients in the context displaying SOFA score data for an individual patient.

As further shown in FIG. 5D, the user interface 500d includes a review notes element 550. The review notes element 550 is a graphical element in the user interface 500d that, when selected, displays the patient's medical charts, treatment notes, and/or any other configured data that has been linked to the review notes element to enable healthcare practitioners to view additional data pertaining to the patient's treatment in the hospital.

As further shown in FIG. 5D, the user interface 500d includes an enter instructions element 552. The enter instructions element 552 is a graphical element in the user interface 500d that, when selected, displays an interface for the healthcare practitioner to enter instructions about the patient's treatment or care. For example, if upon reviewing a patient's predicted SOFA score data displayed on the user interface 500d, the healthcare practitioner may take action to treat or manage a predicted change event associated with an organ system corresponding to the predicted change event by selecting the enter instructions element 552 and entering the instructions. The healthcare practitioner may select the enter instructions element 552 in the user interface 500d to enter instructions such as to prescribe a medication at a specific dosage, to request an EKG, to instruct laboratory work to be performed, or to inform other healthcare professionals to prepare the patient for an urgently needed procedure, such as intubating the patient, placing the patient on a ventilator, or initiating dialysis on the patient.

As further shown in FIG. 5D, the user interface 500d includes current condition data 554. The current condition data 554 indicates the patient's current total SOFA score, current SOFA component scores, and/or current SIRS score which have been estimated for the present time. In other implementations, the current condition data 554 may identify the SOFA and SIRS scores that are determined based on manual input into a patient data records interface that is associated with user interface 500d (not shown).

As further shown in FIG. 5D, the user interface 500d includes a SIRS score prediction graph 556. The SIRS score prediction graph 556 is graph displaying the patient's predicted SIRS score over a specified amount of time into the future. One skilled in the art will appreciate that a variety of graphs types or graph formats may be used to display the SIRS score data in a SIRS score prediction graph 556.

As further shown in FIG. 5D, the user interface 500d includes a total SOFA score prediction graph 558. The total SOFA score prediction graph 558 is graph displaying the patient's predicted total SOFA scores over a specified amount of time into the future. One skilled in the art will appreciate that a variety of graphs types or graph formats may be used to display the total SOFA score data in a total SOFA score prediction graph 558.

As further shown in FIG. 5D, the user interface 500d includes a SOFA component score prediction graph 560. The SOFA component score prediction graph 560 is graph displaying the patient's predicted SOFA component scores over a specified amount of time into the future. In some implementations, the SOFA component score graph 560 may be a plot of all six of a patient's SOFA component scores over one or more specified amounts of time into the future. In other implementations, the SOFA component score graph 560 may be a plot of a single SOFA component score over one or more specified amounts of time into the future. By plotting the SOFA component scores predicted for a patient over one or more specified amounts of time into the future, healthcare practitioners can gain insight into the future condition of the specific organ system that is associated with a particular SOFA component score. The insight gained by viewing plots of the SOFA component scores will assist healthcare practitioners to prepare preventative treatments in advance that are directed at the specific organ systems for which the predicted SOFA component scores correspond. One skilled in the art will appreciate that a variety of graphs types or graph formats may be used to display the SOFA component score data in a SOFA component score prediction graph 560.

In some implementations, a user, such as a healthcare practitioner, may interact with elements of the patient's SOFA component score prediction graph 560 and the user interface 500d will display the physiological parameters typically associated with calculating the particular SOFA component score predicted to occur at some amount of time into the future. For example, consider the user interface 500d shown FIG. 5D where a healthcare practitioner is viewing the SOFA score data for Jack Smith. The user interface 500d displays Jack Smith's SOFA component score data in a graph, such as the SOFA component score prediction graph 560. The graph displays the predicted SOFA component scores that are respectively associated with each SOFA component score organ system as a function of 6, 12, 18, and 24 hours into the future. In some implementations, the healthcare practitioner may further interact with the SOFA component score prediction graph 560 by clicking or selecting an individual SOFA component score organ system identified in the legend (as shown on the left of the SOFA component score prediction graph 452) or clicking or selecting a data point located on or within the graph that corresponds to a specific SOFA organ system component score. In some implementations, the healthcare practitioner may interact with the SOFA component score prediction graph 560 by moving a pointer or mouse cursor over a particular SOFA component score organ system identified in the legend (as shown on the left of the SOFA component score prediction graph 560) or by moving a pointer or mouse cursor over a data point located on or within the graph that corresponds to a specific SOFA component score organ system. Upon selecting or otherwise interacting with the data or graphical elements of the SOFA component score prediction graph 560, the user interface 500d may display physiological parameter values that are associated with the predicted SOFA component score. In some implementations, the predicted physiological parameter value is used in predicting the future SOFA component score. In some implementations, the future SOFA component score is predicted using a model that does not take into account the predicted physiological parameter value. For example, the user interface 500d, upon selecting or clicking the “Respiratory” legend label, may display a data table that is superimposed atop the existing user interface 500d identifying the physiological parameters and parameter values that correspond to Jack Smith's respiratory SOFA component score, such as the measured ratio between Jack Smith's PAO2 (arterial partial pressure) and FIO2 (fraction of inspired oxygen). In some implementations, the user interface 500d, upon selecting or clicking the data point on the graph that is located within graph area corresponding to the respiratory SOFA component score, may transition to or display a new user interface (not shown) where the data identifying with the physiological parameters and parameter values that correspond to Jack Smith's respiratory SOFA component score are displayed.

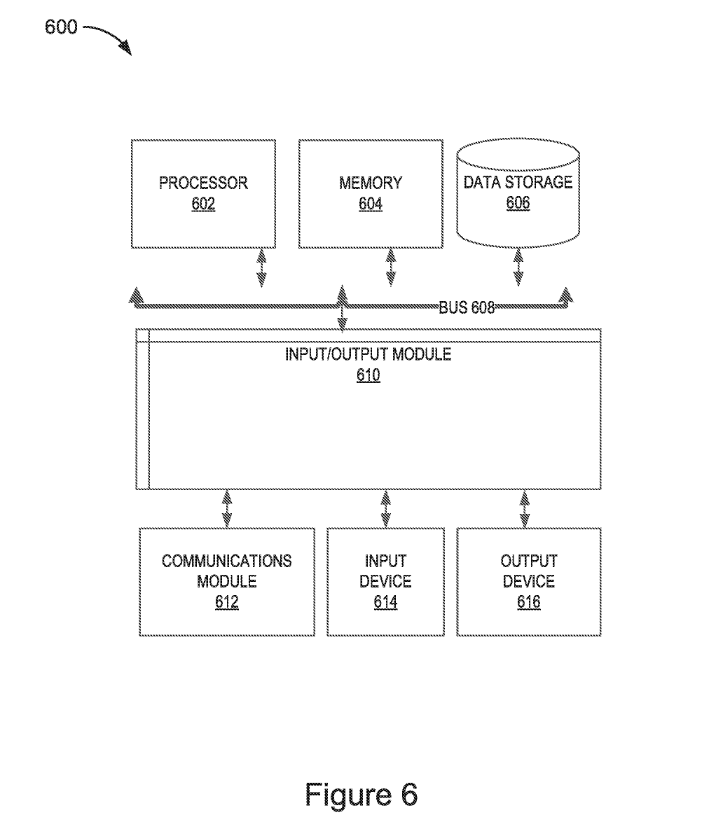

FIG. 6 is a block diagram illustrating an example computer system 600 with which the client 204, server 216, and server 202 of FIGS. 2A and 2B can be implemented. In certain aspects, the computer system 600 may be implemented using hardware or a combination of software and hardware, either in a dedicated server, or integrated into another entity, or distributed across multiple entities.

Computer system 600 (e.g., client 204, server 216, and server 202) includes a bus 608 or other communication mechanism for communicating information, and a processor 602 (e.g., processors 206 and 220) coupled with bus 608 for processing information. According to one aspect, the computer system 600 can be a cloud computing server of an IaaS that is able to support PaaS and SaaS services. According to one aspect, the computer system 600 is implemented as one or more special-purpose computing devices. The special-purpose computing device may be hard-wired to perform the disclosed techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be large-format computer systems, portable computer systems, handheld devices, networking devices or any other device that incorporates hard-wired and/or program logic to implement the techniques. By way of example, the computer system 600 may be implemented with one or more processors 602. Processor 602 may be a general-purpose microprocessor, a microcontroller, a Digital Signal Processor (DSP), an ASIC, a FPGA, a Programmable Logic Device (PLD), a controller, a state machine, gated logic, discrete hardware components, or any other suitable entity that can perform calculations or other manipulations of information.

Computer system 600 can include, in addition to hardware, code that creates an execution environment for the computer program in question, e.g., code that constitutes processor firmware, a protocol stack, a database management system, an operating system, or a combination of one or more of them stored in an included memory (e.g., memory 208 or 222), such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 608 for storing information and instructions to be executed by processors 208 or 220. The processor 602 and the memory 604 can be supplemented by, or incorporated in, special purpose logic circuitry. Expansion memory may also be provided and connected to computer system 600 through input/output module 610, which may include, for example, a SIMM (Single In-Line Memory Module) card interface. Such expansion memory may provide extra storage space for computer system 600, or may also store applications or other information for computer system 600. Specifically, expansion memory may include instructions to carry out or supplement the processes described above, and may include secure information also. Thus, for example, expansion memory may be provided as a security module for computer system 600, and may be programmed with instructions that permit secure use of computer system 600. In addition, secure applications may be provided via the SIMM cards, along with additional information, such as placing identifying information on the SIMM card in a non-hackable manner

The instructions may be stored in the memory 604 and implemented in one or more computer program products, e.g., one or more modules of computer program instructions encoded on a computer readable medium for execution by, or to control the operation of, the computer system 600 and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multi-paradigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, embeddable languages, and xml-based languages. Memory 604 may also be used for storing temporary variable or other intermediate information during execution of instructions to be executed by processor 602.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network, such as in a cloud-computing environment. The processes and logic flows described in this specification can be performed by one or more programmable processors executing one or more computer programs to perform functions by operating on input data and generating output.

Computer system 600 further includes a data storage device 606 such as a magnetic disk or optical disk, coupled to bus 608 for storing information and instructions. Computer system 600 may be coupled via input/output module 610 to various devices (e.g., device 614 or device 616. The input/output module 610 can be any input/output module. Example input/output modules 610 include data ports such as USB ports. In addition, input/output module 610 may be provided in communication with processor 602, so as to enable near area communication of computer system 600 with other devices. The input/output module 602 may provide, for example, for wired communication in some implementations, or for wireless communication in other implementations, and multiple interfaces may also be used. The input/output module 610 is configured to connect to a communications module 612. Example communications modules (e.g., communications module 612 include networking interface cards, such as Ethernet cards and modems).

The components of the system can be interconnected by any form or medium of digital data communication, e.g., a communication network. The communication network (e.g., communication network 214) can include, for example, any one or more of a personal area network (PAN), a local area network (LAN), a campus area network (CAN), a metropolitan area network (MAN), a wide area network (WAN), a broadband network (BBN), the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

For example, in certain aspects, communications module 612 can provide a two-way data communication coupling to a network link that is connected to a local network. Wireless links and wireless communication may also be implemented. Wireless communication may be provided under various modes or protocols, such as GSM (Global System for Mobile Communications), Short Message Service (SMS), Enhanced Messaging Service (EMS), or Multimedia Messaging Service (MMS), CDMA (Code Division Multiple Access), Time division multiple access (TDMA), Personal Digital Cellular (PDC), Wideband CDMA, General Packet Radio Service (GPRS), or LTE (Long-Term Evolution), among others. Such communication may occur, for example, through a radio-frequency transceiver. In addition, short-range communication may occur, such as using a BLUETOOTH, WI-FI, or other such transceiver.

In any such implementation, communications module 612 sends and receives electrical, electromagnetic or optical signals that carry digital data streams representing various types of information. The network link typically provides data communication through one or more networks to other data devices. For example, the network link of the communications module 612 may provide a connection through local network to a host computer or to data equipment operated by an Internet Service Provider (ISP). The ISP in turn provides data communication services through the world wide packet data communication network now commonly referred to as the “Internet”. The local network and Internet both use electrical, electromagnetic or optical signals that carry digital data streams. The signals through the various networks and the signals on the network link and through communications module 612, which carry the digital data to and from computer system 600, are example forms of transmission media.

Computer system 600 can send messages and receive data, including program code, through the network(s), the network link and communications module 612. In the Internet example, a server might transmit a requested code for an application program through Internet, the ISP, the local network and communications module 612. The received code may be executed by processor 602 as it is received, and/or stored in data storage 606 for later execution.

In certain aspects, the input/output module 610 is configured to connect to a plurality of devices, such as an input device 614 (e.g., input device 201) and/or an output device 616 (e.g., output device 202). Example input devices 614 include a keyboard and a pointing device, e.g., a mouse or a trackball, by which a user can provide input to the computer system 600. Other kinds of input devices 614 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Example output devices 616 include display devices, such as a LED (light emitting diode), CRT (cathode ray tube), LCD (liquid crystal display) screen, a TFT LCD (Thin-Film-Transistor Liquid Crystal Display) or an OLED (Organic Light Emitting Diode) display, for displaying information to the user. The output device 616 may comprise appropriate circuitry for driving the output device 616 to present graphical and other information to a user.

According to one aspect of the present disclosure, the client 204, servers 216, 234, and 246 can be implemented using a computer system 600 in response to processor 602 executing one or more sequences of one or more instructions contained in memory 604. Such instructions may be read into memory 604 from another machine-readable medium, such as data storage device 606. Execution of the sequences of instructions contained in main memory 604 causes processor 602 to perform the process steps described herein. One or more processors in a multi-processing arrangement may also be employed to execute the sequences of instructions contained in memory 604. Processor 602 may process the executable instructions and/or data structures by remotely accessing the computer program product, for example by downloading the executable instructions and/or data structures from a remote server through communications module 612 (e.g., as in a cloud-computing environment). In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. For example, some aspects of the subject matter described in this specification may be performed on a cloud-computing environment. Accordingly, in certain aspects a user of systems and methods as disclosed herein may perform at least some of the steps by accessing a cloud server through a network connection. Further, data files, circuit diagrams, performance specifications and the like resulting from the disclosure may be stored in a database server in the cloud-computing environment, or may be downloaded to a private storage device from the cloud-computing environment.

Computing system 600 can include clients and servers. A client and server are generally remote from each other and typically interact through a communication network. The relationship of client and server arises by virtue of computer programs running on the respective computers and having a client-server relationship to each other. Computer system 600 can be, for example, and without limitation, a desktop computer, laptop computer, or tablet computer. Computer system 600 can also be embedded in another device, for example, and without limitation, a mobile telephone, a personal digital assistant (PDA), a mobile audio player, a Global Positioning System (GPS) receiver, a video game console, and/or a television set top box.

The term “machine-readable storage medium” or “computer-readable medium” as used herein refers to any medium or media that participates in providing instructions or data to processor 602 for execution. The term “storage medium” as used herein refers to any non-transitory media that store data and/or instructions that cause a machine to operate in a specific fashion. Such a medium may take many forms, including, but not limited to, non-volatile media, volatile media, and transmission media. Non-volatile media include, for example, optical disks, magnetic disks, or flash memory, such as data storage device 606. Volatile media include dynamic memory, such as memory 604. Transmission media include coaxial cables, copper wire, and fiber optics, including the wires that comprise bus 608. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

### Machine Learning Process Description

The following sections describe details of the a non-limiting example of a machine learning process used to train and generate SOFA score prediction models that are capable of receiving a wide variety of patient data and output SOFA score predictions for a patient at a specified amount of time into the future. To accomplish this goal, correlations between known SOFA scores of patients and the patient's data feature values at the same specified amount of time prior to that known SOFA score were identified. For example, to train a model to predict SOFA values twelve hours into the future, a model is trained based on known SOFA scores and corresponding patient data from twelve hours prior to the SOFA score.

### Data Preparation

In order to perform the machine learning process to train a model capable of predicting patient SOFA scores at specified amounts of time into the future, data associated with patient feature values and SOFA scores in the past was collected and prepared for use in a machine learning process. The data was collected from hospital databases and specifically from the tables representing chart measurements, laboratory measurements, drugs, fluids, microbiology, and cumulative fluids. The patient data from the hospital databases is time-stamped and contains physiological signals and measurements, vital signs, as well as a comprehensive set of clinical data representing such quantitative data as medications taken (amounts, times, and routes), laboratory tests, measurements, and outcomes, feeding and ventilation regimens, and diagnostic assessments. The following tables were used to extract and collect the patient data to be used in the machine learning process:
(1) The encounter table contains the (i) patient id, (ii) date of birth, and (iii) dates and times of admission and discharge.
(2) The assessment table contains charted data for all patients. We recorded (i) the patient id, (ii) the item id, (iii) the time stamp, and (iv) numerical values.
(3) The lab table contains laboratory data for all patients. We recorded the (i) patient id, (ii) the item id, (iii) the time stamp, and (iv) numerical values.
(4) The medication table contains medication data for all patients. We recorded the (i) patient id, (ii) the item id, (iii) the time stamp, and (iv) the medication dose.

As a person of ordinary skill in the art would appreciate that the above tables of data and those in the hospital database correspond to patient data features that have well-known meanings to those of ordinary skill in the art. All patients with sufficient data in the hospital database were included in the data preparation phase of the machine learning process.

While the aforementioned hospital databases may be used as a source of patient data for the machine learning process, the machine learning process may not limited by the exact configuration of the data in the hospital databases or the specific measurements, representations, scales, or units of data included therein. For example, the units that are used to measure a patient data feature that is used in the machine learning process may vary according to the lab or location where the measurement occurs. The standard dose of medication or route of administration may vary between hospitals or hospital systems, or even the particular member of a class of similar medications that are prescribed for a given condition may vary. Mapping of the specific patient data features found in the hospital database to those used in another hospital system are incorporated into the machine learning process to make use of the machine learning process in a different hospital system. For example, if the hospital database measures the weight of patients in pounds and another hospital does so in kilograms, one of ordinary skill in the art would appreciate that it is a simple matter to convert the patients' weights from kilograms to pounds. Likewise, it is straightforward to adjust the predictive formula of the machine learning process to accept kilograms instead of pounds. This sort of mapping between features can also be done between medications that carry out the same functions, but may differ in standard dosages, and/or alternative laboratory measurements that measure the same parameter, vital sign or other aspect in a patient, etc. In addition, rather than mapping patient data feature-to-patient data feature as described in the current paragraph and then using the exemplary models presented here with the newly mapped patient data features, it is straightforward to use the methods taught here to take existing hospital datasets and retrain models in accordance with the techniques of the machine learning process described herein. The models can then be used predictively, in the manner described above as trained SOFA score prediction models. The same patient data feature removal and patient data feature selection methods can be used, or the patient data features found useful here can guide hand-curated patient data feature selection methods. All of this would be apparent to one of ordinary skill in the art.

As indicated above, the models were to be trained by correlating known SOFA scores at known times to patient data at a prior point in time. Accordingly, we began with computing SOFA scores for the patients in the data set. We computed the 6 sub-scores for each patient repeatedly during their ICU stays. The computation of a SOFA score is modeled as a point process during a 24-hour period. FIO2, MAP, and Glasgow Score were extracted from the assessment table. Dopamine, dobutamine, epinephrine, and norepinephrine were extracted from the medication table. Creatinine, platelets, and bilirubin were extracted from the lab table. When multiple sources of a measurement were available during a 24-hour period, the one that had the worst sub-score was used.

For each 24-hour time period for each patient, starting from the beginning of each patient's ICU stay, we computed the SOFA score and recorded it together with the time stamped date and the patient id. We used the time stamps of SOFA score measurements to collect data from the 4 tables, at a time corresponding to 24 hours (the “time point”) prior to each SOFA score computation, using the most recent data nearest the time point for each patient (yet no later than 24 hours prior), but no later than 48 hours than the time stamped date of the SOFA score.

Data were normalized to a mean of zero and standard deviation of one. That is, a normalized version of each datum was created by subtracting the mean for each patient data feature (taken across all occurrences for each feature or measurement type) and divided by the standard deviation (taken across the same distribution).

### Model Selection

To select a prediction model best suited for determining SOFA scores at one or more specified amounts of time into the future, a variety of models were evaluated in the experiment utilizing the patient feature data associated with a SOFA score that was known 24 hours prior to the SOFA scores calculated above. Machine learning was carried out with the following regression models: (i) Linear Regression, (ii) Gradient Boosting Machine, (iii) Linear SVR (linear support vector regression model), (iv) RBF SVR (radial basis function support vector regression model). All methods produced useful results; the epsilon insensitive version of linear SVR was used in the examples shown here. Model and parametric optimization was carried out by running machine learning with different values of method parameters (see C and epsilon, below, as examples). The best regression model was chosen to be the one with the lowest MAE (Mean Absolute Error) on a testing set as defined below, with the models previously fixed on a corresponding training set. Only the best regression model was retained and used in the results presented here.

$$MAE = \frac{1}{N} {\sum }_{I = 1}^{N} \vert {Y}_{i} ( actual ) - {Y}_{i} ( predicted ) \vert$$

where N is the number of samples in the test dataset, Yᵢ(actual) is the actual SOFA score of the ith data sample, Yᵢ(predicted) is the predicted SOFA score of the ith data sample, and the summation over i enumerates the data samples from 1 to N.

An alternative measure to MAE, the root of mean squared error (RMSE), consistently behaved in a manner correspondingly to MAE, in the sense that a model with superior MAE was always found to be superior using the RMSE measure.

In addition, the best model MAE was also compared to the MAE of a naïve regression model that always predicted a constant value for the SOFA score, where the constant was set equal to the average SOFA score of the training set. Such a comparison always obeyed the following inequality: MAE (model)<MAE (naïve model).

Because the Linear SVR performed and generalized very well, the machine learning results presented here use it unless otherwise stated. Although the foregoing is what we used for our work, a person of ordinary skill in the art would readily appreciate that many other machine learning concepts and algorithms could equally be used and applied in the methods, including but not limited to, ordinary least squares regression (OLSR), linear regression, logistic regression, stepwise regression, multivariate adaptive regression splines (MARS), locally estimated scatterplot smoothing (LOESS), ordinal regression, Poisson regression, fast forest quantile regression, Bayesian linear regression, neural network regression, decision forest regression, boosted decision tree regression, artificial neural networks (ANN), Bayesian statistics, case-based reasoning, Gaussian process regression, inductive logic programming, learning automata, learning vector quantization, informal fuzzy networks, conditional random fields, genetic algorithms (GA), Information Theory, support vector machine (SVM), Averaged One-Dependence Estimators (AODE), Group method of data handling (GMDH), instance-based learning, lazy learning, and Maximum Information Spanning Trees (MIST). Moreover, various forms of boosting can be applied with combinations of methods. Some of these learning methods require additional parameters. For the complexity parameter (C) in the linear SVR, in separate runs we used values ranging from 0.0001 to 1000 by powers of ten. For the epsilon (ε) value that governs the sensitivity to loss, we used a grid that ranges from 0.001 to 10 using powers of ten.

In addition to the Linear SVR, the support vector regression model with Radial Basis Function (RBF) kernel was also evaluated. Three separate parameter grids were used to find the model with the best mean absolute error (MAE) of 1.54. The first grid concerns itself with the parameter C (complexity) ranging from 0.0001 to 10 in powers of 10.

Identical grids were also created for GAMMA, a parameter that influences the radius of influence of the support vectors and EPSILON, a parameter that govern the sensitivity of the loss function. Best parameter values are C=1, GAMMA=0.1 and EPSILON=0.1.

For each of these regression models, the model parameters were computed on the basis of the training dataset. For the linear SVR results reported here, the parameters for each resulting model are a set of support vectors, however, for convenience, the equivalent formulation of using one coefficient for each data feature in the model plus a single bias value as in any general linear model was used. A patient data feature is a type of measurement (systolic blood pressure measurement, for example). As shown in the equation below, a linear combination of coefficients (wj) and normalized data features (x_{i,j}=patient_datai,j), together with the bias (b) produces the prediction.

yᵢ=b+w₁*x_{i,1}+w₂*x_{i,2}+ . . . +wⱼ*xᵢⱼ

Each regression model was then used, with its own respective set of parameters obtained from the training dataset (as described above), and was evaluated on the testing dataset and prediction results were expressed by its MAE value. The Linear SVR was selected for its consistently usefully low MAE value, and its robustness to outliers. Several different random combinations of training and test datasets were used to evaluate the reproducibility of the results, which was quite good. This strategy was used to eliminate the possibility that results were due to a serendipitous selection of the test dataset. The SVR regression model results presented here were run with complexity parameter set equal to 10 and epsilon parameter 0.1.

Predictions are made from the Linear SVR model using the above equation: Whose output is the predicted future value of the worst SOFA Score for patient i presenting normalized patient data represented by the vector x_{i,j}, given the model bias parameter b and model coefficients wⱼ corresponding to the normalized patient feature measurements (of which there are numerical features, indexed by j).

For example, using a 5-feature model the values for wⱼ, are w₁=−0.80142 (Base Excess ˜POCT), w₂=−0.741765 (Braden Total Score) w₃=−0.32445 (Platelets [×10⁹/L]), w₄=0.31645 (Urea [mmol/L]), and w₅=−0.6699 (Glasgow Coma Score, total), with j=5. The values for xᵢⱼ for the ith patient are the measurements of the five features (indicated in parentheses in the previous sentence), mean centered and normalized with the corresponding patient data feature values by subtracting the mean and dividing by the standard deviation for each feature. The value for b, the model bias parameter in the equation, is 2.8232. The predicted SOFA score is the resulting yᵢ for patient i, using the equation.

As one of ordinary skill in the art would appreciate, it is straightforward to apply more sophisticated treatments of this value to assign broad classes that describes the severity of a patient's condition. For example, one could use the value directly and map it to categories such as “severe SOFA score range,” “medium SOFA score range” and “mild SOFA score range”. These broad categories may be especially useful to hospitals in taking action on the predictions.

### Qualitative Considerations on Feature Extraction Process

In healthcare, one of the major complexities is the lack of data for certain features. One of the possible solutions is to use the last available value for every missing feature value. Alternatively, the absence of such value may have a clinical meaning that is reflected in the model. Therefore, both solutions are applicable in the sense that, given a sufficiently wide time window, one can limit a collection of values to the most recent feature value within such window. This method, therefore, can be a combination of the two mentioned methods.

Another important consideration is the availability of values for each feature of the entire dataset. When the dataset is split into two separate datasets, the training dataset should have enough values of each feature for the average to be representative of typical values outside of the historical dataset. For instance, given feature A as column vector across all samples, one may count the total number of available values as Nf. The total number of values Nf can be a small fraction of the total number of samples N (Nf/N<<1). To the extent that the number of values is small, it is possible that the average value of such features is not reflective of what it is usually found in outside samples. For these reasons, features that do not have at least 20 values are excluded for predictive purposes. In addition, means and standard deviations are checked for consistency with typical values observed on clinical practices. The exclusion of such features with poor statistics may lead a patient to lose features within the dataset. A patient, therefore, many have not enough features for prediction after some feature elimination. This problem is usually remedied by the following practices: A) the use of limited number of features that exhibit good dependence and or B) the removal of patients from the dataset when the remaining features have not sufficient individual predictive power. The trimming of features need not be fully automatic but can involve the input of an experienced data scientist who strikes a balance between the objectives of including the most information into the model and avoiding bias.

### Feature Selection

During the machine learning process, the patient data was evaluated to select subsets of relevant features for use in a machine learning algorithm used to derive the prediction model capable of determining SOFA scores for specified amounts of time into the future. For example, there is a tremendous amount of data in the patient population dataset, much of which is not necessary or provides little contribution to the predictability of a particular disease, condition, or state for which the prediction model is being trained. Additionally, it is often the case that different particular patients only have available data for different respective subsets of all of the features of the dataset, so that a prediction model based on all of the features of the patient population dataset might not be usable for particular patients or might output suboptimal predictions for the particular patients. An example implementation identifies a plurality of subsets of features within the totality of features of the patient population dataset for which to produce respective prediction models, which can be used to predict a value, e.g., SOFA score, based on data of only the respective subset of features, or even just some of the respective subset of features. Thus, in an example implementation, a computer system is provided with a patient population dataset, from which the system selects a plurality of subsets, each subset being used by a machine learning algorithm, which is applied by the system to the respective subset, to train a new prediction model on the basis of which to predict for a patient onset of multiple organ failures, e.g., SOFA score. Thus, for each selected subset, a respective prediction model can be trained, with each of the trained prediction models being subsequently applied to an individual patient's data with respect to the particular group of features of the subset for which the respective prediction model had been trained.

Thus, according to the example implementations, in a preliminary selection step, a feature selection method is applied to select relevant subsets of patient data features for training respective prediction models. In an example implementations, prior to application of the feature selection method (or, viewed differently, as a first step of the feature selection method), features are initially removed from the dataset based on Bhattacharyya distance. In this process, given a set of predictive patient data features, for each patient data feature we computed the Bhattacharyya distance between the populations of low SOFA scores (<6) and high SOFA scores (>=6). Any patient data feature whose Bhattacharyya distance was found to be less than 0.1 was removed from further consideration. This solution was found to be effective in reducing the number of total patient data features significantly while focusing on those with predictive value. Then, from those features not removed based on the Bhattacharyya distance, the system proceeds to select groups of relevant features to which to apply a machine learning algorithm, where the machine learning algorithm would then generate a respective prediction model based on data values of the selected relevant features.

To reduce the number of features further after the application of the Bhattacharyya criterion, Lasso regression was employed. Lasso (Least Absolute Shrinkage and Selection Operator) is an L1 regularized regression that uses the sum of absolute value of coefficients as a regularization factor. The regularization parameter that regulates the magnitude of the sum of absolute value of coefficients versus the loss minimization objective will be referred to as alpha, based on usage in the scikit-learn Python package implementation.

If alpha=0, Lasso is reduced to standard regression. The larger the magnitude of alpha, the smaller the sum of absolute values of coefficients. Lasso is an effective statistical tool to reduce the number of features in a linear regression problem. Accordingly, by reducing the number of features, the efficiency of the computer performing the machine learning may be improved, e.g., by making more use of available computational resources within the computer and generating results more quickly than before.

The process of feature extraction using the Lasso regression was carried out as follows:

1) A grid of values for the Lasso alpha parameter from 0.01 to 10, in powers of 10, was used sequentially. As the alpha parameter increased in value, the magnitude of many of the coefficients approached zero, thus creating a sparser solution in which some features emerged and others were eliminated (zero coefficient).

2) An alpha parameter from the grid was selected and Lasso was run on the post-Bhattacharyya feature matrix.

3) Two subsets of the feature list were consequently identified: the ones with non-zero coefficients and the remaining ones with zero coefficients.

4) The non-zero coefficient submatrix was then regressed using a linear SVR model iterating over its C and epsilon parameters until a suitable set of C and epsilon parameters was found to maximize MAE. The same set of parameters was then applied to the zero-coefficient submatrix to compute its own MAE value.

Steps (2), (3), and (4) were repeated by changing alpha until the MAE value of the zero-coefficient submatrix had no predictive power. To establish whether a regression model had any predictive power, its MAE value it was compared to the MAE value of a naïve model that always predicted the average SOFA score from the training set. If a model's MAE value was higher than or equal to the naïve model MAE value, then such a model was considered to have no predictive value.

In the case of the Hospital data, the iteration was stopped when the parameter alpha reached the value of 0.1. With an alpha parameter set to 0.1, 23 features were identified in the matrix with non-zero coefficients. The features that comprised the zero-coefficient matrix under Lasso were used to run a linear SVR, which exhibited a MAE value equal to 2.66, slightly higher than the MAE value of the naïve model of 2.60, thus indicating that these features do not offer any relevant predictive power.

As one of ordinary skill in the art would readily appreciate, the above machine learning and feature selection methods were carried out using a particular hospital's database, but the same methods could be utilized on another database from other hospitals to achieve the same results, including identification of primary, secondary and additional features, exemplified here with the hospital database.

### Model Testing and Testing Results

The experimental method also included testing the predictive accuracy of the models selected for use in the machine learning process in order to assess the performance of the model in determining SOFA scores for specified amounts of time into the future based on unseen data. The model testing portion of the experiment included testing the features eliminated by the Lasso procedure using a Linear SVR and producing a MAE value that is inferior in quality to the one by one produced by a naïve regression model. The Lasso non-zero coefficient features were also used to predict SOFA scores using a Linear SVR. To identify the best parameters for this model, we used 2 grids, one for the complexity parameter C and other for the epsilon parameter. All combinations for these two parameters were run to produce the lowest MAE. The original dataset was divided into a training dataset and a test dataset. The training dataset comprised 80% of the original patient data set and was generated by random selection.

The results shown below in Table 1 summarize all the linear SVR bias terms using the 23 features selected using the Lasso procedure.

TABLE 1
Linear SVR model run using the 23 features extracted using
the Lasso procedure. The model MAE value is 1.82. The Linear
SVR model was run using C = 10 and epsilon = 0.1.
Features

age|age
Albumin (g/L)|Albumin
APTT (secs)|APTT
Arterial BP|Mean
Base Excess ~POCT|Base Excess
Bicarbonate (mmol/L)|Bicarbonate
Bilirubin, Conj (umol/L)|Bilirubin, Conj
Braden Total Score|Total Braden
Creatinine (umol/L)|Creatinine
Creatinine (umol/L)|Creatinine_M
FiO2 (%)|FiO2
Glasgow Coma|GCS Total
Haematocrit (%)|Haematocrit (%)
HR|HR
Intake/Output (Net Balance)|Net Balance
NBP|Diastolic
NBP|Mean
NBP|Systolic
Neutrophils (×10⁹/L)|Neutrophils
Phosphate (mmol/L)|Phosphate (mmol/L)
Platelets (×10⁹/L)|Platelets (×10⁹)
RDW (%)|RDW (%)
Urea (mmol/L)|Urea (mmol/L)
Model Bias Parameter, b = 4.1663

To demonstrate the prediction power of the selected features, subsets of 5 features were selected at random out of the 23 predictive features and used in machine learning trials. As in the main model, a Linear SVR model was used to predict SOFA scores found by iterating over the C parameter and epsilon parameter with the same grids used in the main model. The MAE value was only slightly worse than the MAE value with 23 features, indicating that this 5-feature model is usefully predictive. Table 2, shown below, summarizes the features as well as the MAE value obtained from predicting a test dataset.

TABLE 2
Results obtained by training the Linear SVR on 5 features extracted
from the original set of 23 features. MAE value for this model is equal
to 1.83; Parameters are: C = 0.1 and epsilon = 0.1.
Features

Base Excess ~POCT|Base Excess
Braden Total Score|Total Braden
Platelets (×10⁹/L)|Platelets (×10⁹)
Urea (mmol/L)|Urea (mmol/L)
Glasgow Coma|GCS Total
Model Bias Parameter, b = 2.8232

In addition, to the 5 feature model described above, 9 different models were obtained from the original list of 23 features by removing one feature randomly and computing the MAE value using a Linear SVR model. As shown below in Table 3, these models all have nearly as good predictive value as the original 23-feature model, indicating that they are usefully predictive.

TABLE 3
This table contains the features, and MAE values for 9 models
(labeled as MODEL 1-9) obtained by randomly extracting one feature
at a time from the main model. Results show that MAE values are
comparable to the one from the main model. All models were
computed with parameter C = 10 and epsilon = 0.1.
FEATURES

MODEL 1; MAE = 1.892
Albumin (g/L)|Albumin
APTT (sec s)|APTT
Arterial BP|Mean
Base Excess ~POCT|Base Excess
Bicarbonate (mmol/L)|Bicarbonate
Bilirubin, Conj (umol/L)|Bilirubin, Conj
Braden Total Score|Total Braden
Creatinine (umol/L)|Creatinine
Creatinine (umol/L)|Creatinine_M
FiO2 (%)|FiO2
Glasgow Coma|GCS Total
Haematocrit (%)|Haematocrit (%)
HR|HR
Intake/Output (Net Balance)|Net Balance
NBP|Diastolic
NBP|Mean
NBP|Systolic
Neutrophils (×10⁹/L)|Neutrophils
Phosphate (mmol/L)|Phosphate (mmol/
Platelets (×10⁹/L)|Platelets (×10⁹)
RDW (%)|RDW (%)
Urea (mmol/L)|Urea (mmol/L)
Model Bias Parameter, b = 4.17296796
MODEL 2; MAE = 1.891
age|age
APTT (secs)|APTT
Arterial BP|Mean
Base Excess ~POCT|Base Excess
Bicarbonate (mmol/L)|Bicarbonate
Bilirubin, Conj (umol/L)|Bilirubin, Conj
Braden Total Score|Total Braden
Creatinine (umol/L)|Creatinine
Creatinine (umol/L)|Creatinine_M
FiO2 (%)|FiO2
Glasgow Coma|GCS Total
Haematocrit (%)|Haematocrit (%)
HR|HR
Intake/Output (Net Balance)|Net Balance
NBP|Diastolic
NBP|Mean
NBP|Systolic
Neutrophils (×10⁹/L)|Neutrophils
Phosphate (mmol/L)|Phosphate (mmol/
Platelets (×10⁹/L)|Platelets (×10⁹)
RDW (%)|RDW (%)
Urea (mmol/L)|Urea (mmol/L)
Model Bias Parameter, b = 4.16750292
MODEL 3; MAE = 2.041
age|age
Albumin (g/L)|Albumin
Arterial BP|Mean
Base Excess ~POCT|Base Excess
Bicarbonate (mmol/L)|Bicarbonate
Bilirubin, Conj (umol/L)|Bilirubin, Conj
Braden Total Score|Total Braden
Creatinine (umol/L)|Creatinine
Creatinine (umol/L)|Creatinine_M
FiO2 (%)|FiO2
Glasgow Coma|GCS Total
Haematocrit (%)|Haematocrit (%)
HR|HR
Intake/Output (Net Balance)|Net Balance
NBP|Diastolic
NBP|Mean
NBP|Systolic
Neutrophils (×10⁹/L)|Neutrophils
Phosphate (mmol/L)|Phosphate (mmol/
Platelets (×10⁹/L)|Platelets (×10⁹)
RDW (%)|RDW (%)
Urea (mmol/L)|Urea (mmol/L)
Model Bias Parameter, b = 4.07726348
MODEL 4; MAE = 1.896
age|age
Albumin (g/L)|Albumin
APTT (secs)|APTT
Base Excess ~POCT|Base Excess
Bicarbonate (mmol/L)|Bicarbonate
Bilirubin, Conj (umol/L)|Bilirubin, Conj
Braden Total Score|Total Braden
Creatinine (umol/L)|Creatinine
Creatinine (umol/L)|Creatinine_M
FiO2 (%)|FiO2
Glasgow Coma|GCS Total
Haematocrit (%)|Haematocrit (%)
HR|HR
Intake/Output (Net Balance)|Net Balance
NBP|Diastolic
NBP|Mean
NBP|Systolic
Neutrophils (×10 9/L)|Neutrophils
Phosphate (mmol/L)|Phosphate (mmol/
Platelets (×10⁹/L)|Platelets (×10⁹)
RDW (%)|RDW (%)
Urea (mmol/L)|Urea (mmol/L)
Model Bias Parameter, b = 4.16126373
MODEL 5; MAE = 1.912
age|age
Albumin (g/L)|Albumin
APTT (secs)|APTT
Arterial BP|Mean
Bicarbonate (mmol/L)|Bicarbonate
Bilirubin, Conj (umol/L)|Bilirubin, Conj
Braden Total Score|Total Braden
Creatinine (umol/L)|Creatinine
Creatinine (umol/L)|Creatinine_M
FiO2 (%)|FiO2
Glasgow Coma|GCS Total
Haematocrit (%)|Haematocrit (%)
HR|HR
Intake/Output (Net Balance)|Net Balance
NBP|Diastolic
NBP|Mean
NBP|Systolic
Neutrophils (×10⁹/L)|Neutrophils
Phosphate (mmol/L)|Phosphate (mmol/
Platelets (×10⁹/L)|Platelets (×10⁹)
RDW (%)|RDW (%)
Urea (mmol/L)|Urea (mmol/L)
Model Bias Parameter, b = 4.17773078
MODEL 6; MAE = 1.919
age|age
Albumin (g/L)|Albumin
APTT (secs)|APTT
Arterial BP|Mean
Base Excess ~POCT|Base Excess
Bilirubin, Conj (umol/L)|Bilirubin, Conj
Braden Total Score|Total Braden
Creatinine (umol/L)|Creatinine
Creatinine (umol/L)|Creatinine_M
FiO2 (%)|FiO2
Glasgow Coma|GCS Total
Haematocrit (%)|Haematocrit (%)
HR|HR
Intake/Output (Net Balance)|Net Balance
NBP|Diastolic
NBP|Mean
NBP|Systolic
Neutrophils (×10⁹/L)|Neutrophils
Phosphate (mmol/L)|Phosphate (mmol/
Platelets (×10⁹/L)|Platelets (×10⁹)
RDW (%)|RDW (%)
Urea (mmol/L)|Urea (mmol/L)
Model Bias Parameter, b = 4.15443147
MODEL 7: MAE = 1.890
age|age
Albumin (g/L)|Albumin
APTT (secs)|APTT
Arterial BP|Mean
Base Excess ~POCT|Base Excess
Bicarbonate (mmol/L)|Bicarbonate
Braden Total Score|Total Braden
Creatinine (umol/L)|Creatinine
Creatinine (umol/L)|Creatinine_M
FiO2 (%)|FiO2
Glasgow Coma|GCS Total
Haematocrit (%)|Haematocrit (%)
HR|HR
Intake/Output (Net Balance)|Net Balance
NBP|Diastolic
NBP|Mean
NBP|Systolic
Neutrophils (×10⁹/L)|Neutrophils
Phosphate (mmol/L)|Phosphate (mmol/
Platelets (×10⁹/L)|Platelets (×10⁹)
RDW (%)|RDW (%)
Urea (mmol/L)|Urea (mmol/L)
Model Bias Parameter, b = 4.16567936
MODEL 8; MAE = 1.895
age|age
Albumin (g/L)|Albumin
APTT (secs)|APTT
Arterial BP|Mean
Base Excess ~POCT|Base Excess
Bicarbonate (mmol/L)|Bicarbonate
Bilirubin, Conj (umol/L)|Bilirubin, Conj
Creatinine (umol/L)|Creatinine
Creatinine (umol/L)|Creatinine_M
FiO2 (%)|FiO2
Glasgow Coma|GCS Total
Haematocrit (%)|Haematocrit (%)
HR|HR
Intake/Output (Net Balance)|Net Balance
NBP|Diastolic
NBP|Mean
NBP|Systolic
Neutrophils (×10⁹/L)|Neutrophils
Phosphate (mmol/L)|Phosphate (mmol/
Platelets (×10⁹/L)|Platelets (×10⁹)
RDW (%)|RDW (%)
Urea (mmol/L)|Urea (mmol/L)
Model Bias Parameter, b = 4.1595225
MODEL 9; MAE = 1.929
age|age
Albumin (g/L)|Albumin
APTT (secs)|APTT
Arterial BP|Mean
Base Excess ~POCT|Base Excess
Bicarbonate (mmol/L)|Bicarbonate
Bilirubin, Conj (umol/L)|Bilirubin, Conj
Braden Total Score|Total Braden
Creatinine (umol/L)|Creatinine_M
FiO2 (%)|FiO2
Glasgow Coma|GCS Total
Haematocrit (%)|Haematocrit (%)
HR|HR
Intake/Output (Net Balance)|Net Balance
NBP|Diastolic
NBP|Mean
NBP|Systolic
Neutrophils (×10⁹/L)|Neutrophils
Phosphate (mmol/L)|Phosphate (mmol/
Platelets (×10⁹/L)|Platelets (×10⁹)
RDW (%)|RDW (%)
Urea (mmol/L)|Urea (mmol/L)
Model Bias Parameter, b = 4.16776477

The results shown above indicate that different features possess different predictive capabilities. One way to measure the predictive power or capability of each feature is to compare a model's performance before and after the removal of the specific feature from the feature training set for which the model was trained. The model features determined to have the greatest predictive power (ranked highest to lowest) are the Glasgow Coma Score (highest), platelet values, creatinine values, and the Braden Score (lowest). When these four features are unavailable, the model MAE performance worsens by 23.6% to 2.25. In addition, when the feature set excludes features that are not traditionally used to determine SOFA score calculations, the features determined to have the greatest predictive power (ranked highest to lowest) are the Braden Score (highest), Albumin levels, heart rate, and age (lowest). When these four features are unavailable, the model MAE performance worsens by 6.6% to 1.94.

Accordingly, it is desirable for a predicted total SOFA score to be determined using at least three of the four physiological parameters which have been determined to have the greatest predictive power. For example, a model will have a greater predictive performance when the current values of a patient's Braden Score and the current values for at least two out of three of the patient's Glasgow Coma scale, platelet levels, and creatinine levels are used as model input to predict the patient's total SOFA score. In some implementations, all four of the physiological parameters that are traditionally used for total SOFA score prediction may be used as inputs to the total SOFA score prediction model.

It is also desirable for a predicted total SOFA score to be determined using at least three of the four physiological parameters which have the greatest predictive powers that are not traditionally used for total SOFA score determination. For example, a model will tend to have greater predictive performance when the current values of a patient's Braden Score and the current values for at least two out of three of the patient's albumin level, heart rate, and age are used as model inputs to predict the patient's total SOFA score. In some implementations, all four of these physiological parameters are used as inputs to the SOFA score prediction model.

While indicated above that certain numbers of the most predictive features are desirable to include as inputs to the total SOFA score prediction model, it is understood that different training data sets might identify different sets of features as being more predictive. For example, certain features may be more predictive across certain demographics or in certain geographic regions than in others. Accordingly, in some implementations, a total SOFA score prediction model may include fewer of the features indicated above as being the most predictive as inputs without departing from the scope of the disclosure.

The above experimental data demonstrates the capability of machine learning techniques to be used in predicting total SOFA scores at times in the future. Similar model training processes can likewise be used to train models to predict SOFA component scores and specific physiological parameter values at different amounts of time into the future.

### Physiological Parameter Prediction

As indicated above, a similar model training process to that described above can be used to train models to predict individual physiological parameters. These models may be used on a stand-alone basis or to support predictions made by a model trained to compute a total SOFA score or SOFA component scores. Descriptions of five models and the evaluation results are presented below:

### Bilirubin Prediction Model (Model A)

Bilirubin is measured in μmol/L with a typical range between 20 and 200+. One suitable model for use in predicting bilirubin is an RBF (radial basis function) SVR with parameters C=1,Gamma=0.1 and Epsilon=0.1. The feature selection was carried out as described above using Linear Lasso. The validation set carried out a MAE value=24.92. One example set of features found to be effective using the RBF SVR is listed below. Each feature is listed in the format of “feature measurement|feature name”.

| | |
|---|---|
| Vt (Set)\|Tidal Volume | Albumin (g/L)\|Albumin |
| Arterial BP\|Diastolic | MCHC\|MCHC |
| RDW (%)\|RDW (%) | Bilirubin, Total (umol/L)\|Bilirubin, Tot |
| Carbon Dioxide (mmol/L)\|CO2 | Uric Acid (mmol/L)\|Uric Acid |
| INR\|INR | Bilirubin, Conj (umol/L)\|Bilirubin, Conj |
| Anion Gap (mmol/L)\|Anion Gap | Bilirubin, Unconj (umol/L)\|Bilirubin, |
| Neutrophils (%)\|Neutrophils (%) | Unconj |
| ALP (U/L)\|ALK | Creatinine (umol/L)\|Creatinine |
| Sodium (mmol/L)\|Sodium | ALT (U/L)\|ALT (U/L) |
| (mEq/L) | |

### Creatinine Prediction Model (Model B)

Creatinine is measured in μmol/L with a typical range between 110 and 400+. One suitable model for use in predicting creatinine is an RBF (radial basis function) SVR with parameters C=1,Gamma=0.1 and Epsilon=0.1. The feature selection was carried out as described above using Linear Lasso. The validation set carried out a MAE value=44.70. One example set of features found to be effective using the RBF SVR is listed below. Each feature is listed in the format of “feature measurement|feature name”.

| | |
|---|---|
| Glasgow Coma\|GCS Total | POCT Glucose-Performa (mmol/L)\|Glucose |
| Apache II\|Apache Se Creati | Monocytes (%)\|Monocytes (%) |
| FiO2 (%)\|FiO2 | Chloride (mmol/L)\|Cl |
| HR\|HR | MPV(fL)\|MPV (fL) |
| Temperature (° C.)\|Temp (C) | Lymphocytes (%)\|Lymphocytes (%) |
| Temp F\|Temp (F) | Bilirubin, Conj (umol/L)\|Bilirubin, Conj |
| Blood sugar\|Blood sugar | Neutrophils (×10 9/L)\|Neutrophils |
| NBP\|Systolic | AST (U/L)\|AST(U/L) |
| Magnesium (mmol/L)\|Mg | eGFR\|Value |
| Lactate (mmol/L)\|Lactate (mmol/L) | Bilirubin, Unconj (umol/L)\|Bilirubin, Unconj |
| Potassium (mmol/L)\|Potassium | Creatinine (umol/L)\|Creatinine |
| Anion Gap (mmol/L)\|Anion Gap | Base Excess (mmol/L)\|Base Excess |
| pCO2 ~POCT\|Arterial pCO2 | |
| Neutrophils (%)\|Neutrophils (%) | |
| Calcium (mmol/L)\|Ca | |
| Anion Gap (mmol/L) ~POCT\|Anion Gap | |
| Creatinine (umol/L)\|Creatinine_M | |

### Platelet Prediction Model (Model C)

Platelets are measured in 10**3/L with a typical range between 10 and 200+. One suitable model for use in predicting creatinine is an RBF (radial basis function) SVR with parameters C=1, Gamma=0.01 and Epsilon 0.001. The feature selection was carried out as described above using Linear Lasso. The validation set carried out a MAE value=11.35. One example set of features found to be effecting using the RBF SVR is listed below. Each feature is listed in the format of “feature measurement|feature name”.

| | |
|---|---|
| NBP\|Diastolic | Neutrophils (%)\|Neutrophils (%) |
| CaO2 (ml/dl)\|CaO2 | Calcium (mmol/L)\|Ca |
| Glasgow Coma\|GCS Total | ALP (U/L)\|ALK |
| Weight (Kg) (Daily)\|Weight | MCH (pg)\|MCH (pg) |
| Urine Output\|ml/hr last 24 H | O2 Saturation ~POCT\|Arterial SaO2 |
| O2 (L/min)\|O2 Flow | Phosphate (mmol/L)\|Phosphate (mmol/ |
| Blood sugar\|Blood sugar | Platelets (×10 9/L)\|Platelets (×10 9 |
| AaDO2\|Arterial pO2 | pH ~POCT\|Arterial pH |
| Urinary Catheter\|Duration | POCT Glucose-Performa (mmol/L)\|Glucose |
| NBP\|Systolic | MCHC\|MCHC |
| Magnesium (mmol/L)\|Mg | pO2 (mmHg)\|Arterial pO2 |
| Lactate (mmol/L)\|Lactate (mmol/L) | MPV(fL)\|MPV (fL) |
| Creatinine Kinase (U/L)\|CK_M | Urea (mmol/L)\|Urea (mmol/L) |
| LUC (%)\|LUC (%) | “Bilirubin, Conj (umol/L)\|Bilirubin, Conj” |
| RDW (%)\|RDW (%) | WBC (×10 9/L)\|WBC (×10 9/L) |
| Anion Gap (mmol/L)\|Anion Gap | APTT (secs)\|APTT |
| CKMB (ug/L)\|CK-MB | “Calcium, Corrected\|Value” |
| Base Excess ~POCT\|Base Excess | Base Excess (mmol/L)\|Base Excess |
| Haemoglobin (g/dL)\|Haemoglobin (g/dL) | |

### Mean Arterial Blood Pressure (MAP) Prediction Model (Model D)

Mean Arterial Blood Pressure (MAP) is measured in mm/Hg with a typical range between 10 and 200+. One model suitable for use in predicting values associated with the Mean Arterial Blood Pressure is an RBF (radial basis function) SVR with parameters C=1,Gamma=0.01 and Epsilon 0.001. The feature selection was carried out as described above using Linear Lasso. The validation set carried out a MAE value=6.48. One sample set of features found to be effective using the RBF SVR is listed below. Each feature is listed in the format of “feature measurement|feature name”.

| | |
|---|---|
| NBP\|Diastolic | AaDO2\|AaDO2 |
| Urine Output\|Last 24 H (ml) | NBP\|Systolic |
| Glasgow Coma\|GCS Total | APTT (secs)\|APTT |
| Arterial BP\|Systolic | RDW (%)\|RDW (%) |
| Weight (Kg) (Daily)\|Weight | INR\|INR |
| PIP\|PIP | Potassium (mmol/L)\|Potassium |
| Intake/Output (Net Balance)\|Net Balance | Sodium ~POCT\|Sodium (mEq/L) |
| Total Rate (Vent + Pt)\|Respiration Rate | Std HCO3 ~POCT\|Arterial HCO3 |
| FiO2 (%)\|FiO2 | Red Blood Cells (×10 9/L)\|RBC (×10 12/L) |
| HR\|HR | Sodium (mmol/L)\|Sodium (mEq/L) |
| Vt (Set)\|Tidal Volume | MCV (fL)\|MCV (fL) |
| Arterial BP\|Diastolic | pH ~POCT\|Arterial pH |
| NBP\|Mean | Albumin (g/L)\|Albumin |
| Arterial BP\|Mean | MCHC\|MCHC |
| Apache II\|Apache Mean ABP | HB ~POCT (g/dL)\|Hb POCT (g/dL) |
| Pulse Pressure Value\|PP Value | MPV(fL)\|MPV (fL) |
| Urinary Catheter\|Duration | Platelets (×10 9/L)\|Platelets (×10 9 |
| | Urea (mmol/L)\|Urea (mmol/L) |

Glasgow Coma Score Prediction Model (Model E)

The ability to predict future GCS values can facilitate additional use cases since the GCS may also be used alone (e.g., with stroke patients) or as a component of other composite scores. Glasgow score is defined between 3 and 16. The validation set carried out a MAE value=1.04. One suitable model for use in predicting the Glasgow Coma Score is a neural network MLP (Multilayer Perceptron) that uses all possible features. The feature extraction is done at the level of the first hidden layer with 200 nodes.

MLPRegressor(activation=‘tanh’, hidden_layer_sizes=(200,200),solver=“lbfgs”.

### Sofa Score Prediction Use Cases

In addition to the general patient triage applications described above the process can be used or adapted for the following additional use cases. The following describes one or more implementations of applying the prediction model generated in the machine learning process to patient data in order to output SOFA score predictions for a patient at specified amounts of time into the future.

The next-day SOFA score for a given patient can be estimated with good accuracy. The examples described above show methods for building predictive models for the SOFA score using a relatively small number of features (patient data measurements, observations, etc.) pared down from the much larger number of data types that may or may not be available for a particular patient in a hospital database, such as the hospital database. The models developed and shown here can be used directly to make predictions for hospital patients. One merely needs to acquire measurements of data for a particular patient corresponding to the features in the model, normalize them as shown here, use the model parameters (bias b and coefficients wⱼ), and apply the linear regression formula to produce a SOFA score for the patient at the time point indicated by the model (here 24 hours in advance). If the model score is negative, it is truncated to zero. If the predicted SOFA score is larger than 24, it is truncated to 24. As described above, the SOFA score can be used in a multitude of ways to assign a less fined grained multiclass classification of the severity of the SOFA Score.

The unexpectedly high predictive ability for SOFA score of the methods have been shown in this application, for example, by the low MAE values and other predictive result determinations. The unexpectedly high predictive accuracy with relatively small sets of feature measurements has also been shown in this application. For example, using the 5 features, the method resulted in an in a MAE value equal to 1.83, only 1% higher than the model that uses all 23 features. The model 23 features of the main model were applied to the 80% of data designated as training data according to the above method to determine the next 24 hours SOFA score using those features, and the MAE value of 1.82 was determined against the 20% test data relative to those same features by computing the absolute difference between the actual SOFA score and the predicted one for all patients in the dataset, as a person of ordinary skill in the art would appreciate.

Rather than use the precise models presented here directly, one can use the methods here to produce new models, using available hospital data from the above database and/or databases other than that identified herein (for example, historical or retrospective data from the previous few weeks, months, or years at the same or similar hospital or hospital system) and apply the methods to identify feature sets and models, and then to apply them as described here. The methods shown here can be used to prepare the data, select features, and carry out machine learning to produce models and evaluate the predictive ability of those models. The methods shown here can then be used to apply those models to make predictions on new patients using current measurements on those new patients.

For example, with regard to a patient who walks in the door of a hospital for assessment, the method can be applied in the following manner relative to the hospital database features (or features from another database, as applicable). The patient's data can be obtained for the various features over the course of time and in the ordinary course of the patient's stay in the hospital. To the extent that the obtained measurements match any of the above models and their parameter sets, the method and the above models can be applied to the patient's features to determine a patient's SOFA score 24 hours in advance. For example, if one has the measurement corresponding to Glasgow Coma score, one can make a prediction using that patient measurement, normalizing, and applying the coefficient and bias from the table to produce an estimate of the SOFA score 24 hours after the measurement was taken. If the model predicts the onset of a high SOFA score, the hospital can advantageously begin treating the patient for such condition, thus saving time and money as compared to waiting for the more dire situation in which such a high SOFA score has already occurred.

Alternatively, as features of the patient are ascertained during his or her stay at the hospital, new models can be created based on those features as described above (using the hospital database or another database and its features, as applicable) and tested for predictive ability in terms of future SOFA scores in the patient. That is, if a patient's measurements correspond to a combination of features for which a model has not previously been trained, one can use methods described here to train such a model using historical (past) data with those features only. One can test those models on historical (past) testing set data as described here. One can assess the MAE and other metrics quantifying the performance of the model on patients in the testing set as described here. Finally, one can then apply the model to the new patient or to new patients as described here. In this case, as in the others described here, treatment of the patient or patients for high predicted SOFA scores can be advantageously initiated before the patient reaches such levels if the model predicts the scores 24 hours in advance. Alternatively, a hospital could base the decision on whether to begin treatment for high predicted SOFA scores in a less severe patient based on the relative predictive results of the model (e.g., such treatment would begin in a less severe patient with high predicted future SOFA scores that the model predicts 24 hours in advance). For example, a hospital may decide to begin treatment if the predicted SOFA score exceeds a given threshold.

On the other hand, a patient could walk in the door of a hospital that measures features in a manner that is different from that of the Hospital database (or some features are the same and one or more features are different in terms of units or a different measurement that is used to assess the same aspect of a patient or a different dose of the same or different medication is used to treat the same aspect of a patient, etc.). First, the features that are different than the hospital features can be mapped to the hospital features by recognizing the similarity of what the measurement achieves (for example, different ways of measuring blood urea, Glasgow Coma score, Braden score, creatinine, and other features that are part of the 23 features of the main model).

The above models or new models can be used to predict SOFA scores at a given time in the future, with advantageous early treatment being applied as set forth in the above paragraph. For example, simply developing new normalization parameters for new measurements using the method for how normalization was carried out here would allow new measurements to be incorporated into the models presented here. Alternatively, if there is an existing database for the particular hospital that uses features other than Hospital features (or a mixture of Hospital features and other features), new models can be prepared to select features from that database that can be used to predict in advance SOFA scores as described herein. As described here, features would be eliminated and selected, data normalized, and models built and tested using the methods disclosed in this application. The patient's data then can be obtained for these various features over the course of time and in the ordinary course of the patient's stay in the hospital. These new models prepared using the hospital's database can be applied to the patient's features to predict SOFA scores in advance. Patient measurements can be normalized, inserted into the model, and the model would then make a prediction on the next 24-hour SOFA score. Alternatively, as features of the patient are ascertained (measured) during his or her stay at the hospital, new models can be created based on those features in accordance with the methods described above (using the hospital's database) and tested for predictive accuracy of SOFA scores for the patient using historical (past) patients at the same or similar hospital or hospital system, as described above. New measurements for the patient can be used in these new models to predict in advance the SOFA score in the new patient. In either case, treatment of the patient for high levels of predicted SOFA score can be advantageously initiated before such condition occurs if the model predicts SOFA scores in advance. Alternatively, a hospital could base the decision on whether to begin treatment for a high level of SOFA score in a less severe patient based on the predicted value of SOFA score by such model.

In another example implementation, a hospital, medical center, or health care system maintains multiple models simultaneously. The measurements for a patient can be input into multiple models to obtain multiple values for SOFA score at the same or different times in the future. These different predicted values can be combined to develop an aggregate predicted SOFA score and an action plan can be developed accordingly. For example, the different models could predict whether the SOFA score will be above a certain threshold within a given timeframe, and the aggregate prediction could be made based on the outcome of this voting scheme. The voting can be unweighted (each model receives an equal vote), or weighted based on the accuracy or other quantitative metric of the predictive abilities of each model (with more accurate or higher quality models casting a higher proportional vote).

Further, in another example implementation, the predicted SOFA score can be used in conjunction with other predicted scores (e.g., SIRS score) to generate a master score. The master score can be a weighted composite of the SOFA score and the other predicted scores. In an implementation, the weighting applied to each predicted score will vary depending on the patient. For example, one could take a weighted linear combination of various different scores, such as SOFA scores, APACHE II scores, and/or the probability of SIRS in the next 48 hours, to create a new aggregate score that might have more usefulness in a hospital setting. A linear combination is computed by taking each score and multiplying it by a coefficient, and adding all of these score-coefficient products together.

In yet another example implementation, one can use multiple models and base a prediction on the first one for which a sufficient number of measurements have been obtained for the current patient. In another implementation, the parameters for a model can be re-computed (updated) using additional data from the greater number of historical patients available as time progresses. For example, every year, every month, every week, or every day, an updated database of historical (past) patients can be used to retrain the set of models in active use by creating a training and testing dataset from the available past data, training the models on the training data, and testing them to provide quantitative assessment on the testing data as described here.

As used in this specification of this application, the terms “computer-readable storage medium” and “computer-readable media” are entirely restricted to tangible, physical objects that store information in a form that is readable by a computer. These terms exclude any wireless signals, wired download signals, and any other ephemeral signals. Storage media is distinct from but may be used in conjunction with transmission media. Transmission media participates in transferring information between storage media. For example, transmission media includes coaxial cables, copper wire and fiber optics, including the wires that comprise bus 608. Transmission media can also take the form of acoustic or light waves, such as those generated during radio-wave and infra-red data communications. Furthermore, as used in this specification of this application, the terms “computer”, “server”, “processor”, and “memory” all refer to electronic or other technological devices. These terms exclude people or groups of people. For the purposes of the specification, the terms display or displaying means displaying on an electronic device.

In one aspect, a method may be an operation, an instruction, or a function and vice versa. In one aspect, a clause or a claim may be amended to include some or all of the words (e.g., instructions, operations, functions, or components) recited in other one or more clauses, one or more words, one or more sentences, one or more phrases, one or more paragraphs, and/or one or more claims.

To illustrate the interchangeability of hardware and software, items such as the various illustrative blocks, modules, components, methods, operations, instructions, and algorithms have been described generally in terms of their functionality. Whether such functionality is implemented as hardware, software or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for”.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted that the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A computer-implemented method for predicting sequential organ failure assessment (SOFA) scores using machine learning, the method comprising:
receiving patient data, the patient data including a plurality features associated with each of one or more patients;
processing the plurality of features for each patient using a plurality of SOFA score prediction models derived from at least one machine learning process to output a plurality of respective predicted SOFA scores, wherein a first of the prediction models has been trained to output a first SOFA component score for a first amount of time into the future and a second of the prediction models has been trained to output a second SOFA component score for the first amount of time into the future; and
outputting on a graphical user interface, for each of the patients, a total SOFA score and at least one of the first SOFA component score and the second SOFA component score predicted for the respective patient.

2. The computer-implemented method of claim 1, further comprising determining the total SOFA score for each patient via a third prediction model trained to output total SOFA scores for the first amount of time into the future.

3. The computer-implemented method of claim 1, further comprising calculating the total SOFA score for each patient by summing the values of six SOFA component scores for a given patient for first amount of time into the future, wherein each of the SOFA component scores is associated with a different organ system.

4. The computer-implemented method of claim 1, further comprising determining a second total SOFA score for each patient by via a fourth prediction model trained to output total SOFA scores for a second amount of time into the future.

5. The computer-implemented method of claim 1, further comprising:
processing the patient data for each patient using a SIRS score prediction model derived from a machine learning process to output a predicted SIRS score, wherein the SIRS score prediction model has been trained to output a value indicating the likelihood of a patient having at least two SIRS symptoms the first amount of time into the future; and
outputting on a graphical user interface, for each of the patients, the SIRS score along with the total SOFA score and at least one of the first SOFA component score and the second SOFA component score predicted for the respective patient.

6. The computer-implemented method of claim 1, wherein the each of the first and second SOFA component scores correspond to a different one of a respiratory organ system, a cardiovascular organ system, a hepatic organ system, a coagulation organ system, a renal organ system, and a neurological organ system.

7. The computer-implemented method of claim 1, further comprising outputting on the graphical user interface an indication for at least one patient of any SOFA component scores predicted to exceed a threshold value, an identification of the organ system associated with the SOFA component score exceeding the threshold value, and the amount of time in the future at which the SOFA component score is predicted to exceed the threshold.

8. The computer implemented method of claim 1, further comprising processing a subset of the plurality of features to estimate a current value of a physiological parameter for a patient, wherein the physiological parameter is a physiological parameter used in calculating a current SOFA component score.

9. The computer implemented method of claim 1, further comprising processing a subset of the plurality of features to predict a future value of a physiological parameter for a patient score for the first amount of time into the future, wherein the physiological parameter is a physiological parameter traditionally used in calculating a SOFA component score.

10. The computer-implemented method of claim 1, the method further comprising outputting on the graphical user interface a list of patients for whom any SOFA component score is predicted to exceed a threshold value the first amount of time in the future.

11. A system for predicting sequential organ failure assessment (SOFA) scores using machine learning, the system comprising:
a memory storing computer-readable instructions and a plurality of SOFA score prediction models; and
a processor, the processor configured to execute the computer-readable instructions, which when executed carry out the method comprising:
receiving patient data, the patient data including a plurality features associated with one or more patients;
processing the plurality of features for each patient using a plurality of SOFA score prediction models derived from at least one machine learning process to output a plurality of respective predicted SOFA scores, wherein a first of the prediction models has been trained to output a first SOFA component score for a first amount of time into the future and a second of the prediction models has been trained to output a second SOFA component score for the first amount of time into the future; and
outputting on a graphical user interface, for each of the patients, a total SOFA score and at least the first SOFA component score and the second SOFA component score predicted for the respective patient.

12. The system of claim 11, wherein the memory further stores computer-readable instructions, which when executed cause the processor to determine the total SOFA score for each patient via a third prediction model trained to output total SOFA scores for the first amount of time into the future.

13. The system of claim 11, wherein the memory further stores computer-readable instructions, which when executed cause the processor to calculate a total SOFA score for each patient by summing the values of six SOFA component scores for the patient for first amount of time into the future, wherein each of the SOFA component scores is associated with a different organ system.

14. The system of claim 11, wherein the memory further stores computer-readable instructions, which when executed cause the processor to determine a second total SOFA score for each patient by via a fourth prediction model trained to output total SOFA scores for a second amount of time into the future.

15. The system of claim 11, wherein the memory further stores computer-readable instructions, which when executed cause the processor to carry out the method further comprising:
processing the patient data for each patient using a plurality of SIRS score prediction models derived from at least one machine learning process to output a predicted SIRS score, wherein the plurality of SIRS score prediction models have been trained to output a SIRS score for one or more amounts of time into the future; and
outputting on a graphical user interface, for each of the patients, the SIRS score in addition to the total SOFA score, the first SOFA component score and/or the second SOFA component score predicted for the respective patient.

16. The system of claim 11, wherein the each of the first and second SOFA component scores correspond to a different one of a respiratory organ system, a cardiovascular organ system, a hepatic organ system, a coagulation organ system, a renal organ system, and a neurological organ system.

17. The system of claim 11, wherein the memory further stores computer-readable instructions, which when executed cause the processor to output on the graphical user interface the total SOFA score and the first and second SOFA component scores and displaying an indication of the first and second SOFA component scores exceeding a threshold value, wherein the graphical output identifies the organ system associated with the SOFA component score exceeding the threshold value.

18. The system of claim 11, wherein the memory further stores computer-readable instructions, which when executed cause the processor to output on the graphical user interface the total SOFA score and the first and second SOFA component scores and displaying an indication identifying a list of patients whose total SOFA score and first or second SOFA component scores exceeds a threshold value.

19. The system of claim 11, wherein the memory further stores computer-readable instructions, which when executed cause the processor to carry out the method comprising processing a subset of the plurality of features to estimate a current value of a physiological parameter for a patient, wherein the physiological parameter is a physiological parameter used in calculating a current SOFA component score.

20. The system of claim 11, wherein the memory further stores computer-readable instructions, which when executed cause the processor to carry out the method comprising processing a subset of the plurality of features to predict a future value of a physiological parameter for a patient score for the first amount of time into the future, wherein the physiological parameter is a physiological parameter traditionally used in calculating a SOFA component score.

21. A system for predicting a total sequential organ failure assessment (SOFA) score, the system comprising:
a memory storing computer-readable instructions and a total SOFA score prediction model; and
a processor, the processor configured to execute the computer-readable instructions, which when executed carry out the method comprising:
receiving patient data, the patient data including a plurality features associated with each of one or more patients;
processing the plurality of features for each patient using a total SOFA score prediction model derived from at least one machine learning process to output a predicted total SOFA score for the patient for a first amount of time into the future, wherein the total SOFA score prediction model takes as input the patient's current values of at least three physiological parameters, including a Braden Score and at least two of Glasgow Coma Scale, platelet level, and creatinine level; and
outputting on a graphical user interface, for each of the patients, the total SOFA score predicted for the respective patients for the first amount of time into the future.

22. The system of claim 21, wherein the total SOFA score prediction model takes as input the patient's current values of a Braden Score, platelet level, creatinine level, and the Glasgow Coma Scale.

23. The system of claim 21, wherein the total SOFA score prediction model further takes as input the patient's current values of at least two of albumin level, heart rate, and age.

24. The system of claim 21, wherein the total SOFA score prediction model further takes as input the patient's current values of albumin level, heart rate, and age.

25. The system of claim 21, wherein the total SOFA score prediction model comprises a support vector regression model.

26. The system of claim 21, wherein the total SOFA score prediction model comprises a radial basis function support vector regression model.

27. The system of claim 21, wherein the memory further stores computer-readable instructions, which when executed cause the processor to carry out the method further comprising determining the total SOFA score for each patient via a second prediction model trained to output a total SOFA score for a second amount of time into the future, different than the first amount of time into the future.

28. The system of claim 21, wherein the memory further stores computer-readable instructions, which when executed cause the processor to carry out the method further comprising determining a future value of one or more SOFA component scores for each patient predicted for the first amount of time into the future.

29. The system of claim 21, wherein the memory further stores computer-readable instructions, which when executed cause the processor to carry out the method further comprising, for each patient, displaying a SOFA component score predicted for the first amount of time into the future.

30. The system of claim 29, wherein the memory further stores computer-readable instructions, which when executed cause the processor to carry out the method further comprising outputting on the graphical user interface an indication of at least one predicted physiological parameter value associated with the predicted SOFA component score.

31. A computer-implemented method for predicting a total sequential organ failure assessment (SOFA) score, the method comprising:
receiving patient data, the patient data including a plurality features associated with each of one or more patients;
processing the plurality of features for each patient using a total SOFA score prediction model derived from at least one machine learning process to output a predicted total SOFA score for the patient for a first amount of time into the future, wherein the total SOFA score prediction model takes as input the patient's current values of at least three physiological parameters, including a Braden Score and at least two of Glasgow Coma Scale, platelet level, and creatinine level; and
outputting on a graphical user interface, for each of the patients, the total SOFA score predicted for the respective patients for the first amount of time into the future.

32. The computer-implemented method of claim 31, wherein the total SOFA score prediction model takes as input the patient's current values of a Braden Score, platelet level, creatinine level, and the Glasgow Coma Scale.

33. The computer-implemented method of claim 31, wherein the total SOFA score prediction model further takes as input the patient's current values of at least two of albumin level, heart rate, and age.

34. The computer-implemented method of claim 31, wherein the total SOFA score prediction model further takes as input the patient's current values of albumin level, heart rate, and age.

35. The computer-implemented method of claim 31, wherein the total SOFA score prediction model comprises a support vector regression model.

36. The computer-implemented method of claim 31, wherein the total SOFA score prediction model comprises a radial basis function support vector regression model.

37. The computer-implemented method of claim 31, further comprising determining the total SOFA score for each patient via a second prediction model trained to output a total SOFA score for a second amount of time into the future, different than the first amount of time into the future.

38. The computer-implemented method of claim 31, further comprising determining a future value of one or more SOFA component scores for each patient predicted for the first amount of time into the future.

39. The computer-implemented method of claim 31, further comprising, for each patient, displaying a SOFA component score predicted for the first amount of time into the future.

40. The computer-implemented method of claim 39, further comprising outputting on the graphical user interface an indication of at least one predicted physiological parameter value associated with the predicted SOFA component score.

41. A computer readable storage medium containing program instructions for causing a computer to predict sequential organ failure assessment (SOFA) scores using machine learning performed by the method of:
receiving patient data, the patient data including a plurality features associated with one or more patients;
processing the plurality of features for each patient using a plurality of SOFA score prediction models derived from at least one machine learning process to output a plurality of respective predicted SOFA scores, wherein a first of the prediction models has been trained to output a first SOFA component score for a first amount of time into the future and a second of the prediction models has been trained to output a second SOFA component score for the first amount of time into the future; and
outputting on a graphical user interface, for each of the patients, a total SOFA score and at least the first SOFA component score and the second SOFA component score predicted for the respective patient.
